(19) United States
(12) Patent Application Publication
(10) Pub. No.: US 2007/0099911 A1
(43) Pub. Date: May 3, 2007

(54) Pyrroloquinolinone derivatives as 5-hydroxytryptamine-6 ligands

(75) Inventors: Michael Gerard Kelly, Thousand Oaks, CA (US); Steven Edward Lenicek, Plainsboro, NJ (US); Yvette Latko Palmer, Yardley, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/588,436

(22) Filed: Oct. 27, 2006

Related U.S. Application Data
(60) Provisional application No. 60/731,137, filed on Oct. 28, 2005.

(51) Int. Cl. A61K 31/541; A61K 31/5377; A61K 31/496; A61K 31/519; A61K 31/4745; C07D 487/02; C07D 471/02

(52) U.S. Cl. 514/227.8; 514/234.2; 514/252.16; 514/267; 514/291; 544/60; 544/122; 544/250; 544/361; 514/253.03; 546/81

(57) ABSTRACT

The present invention provides a compound of formula I and the use thereof in the therapeutic treatment of a central nervous system disorder related to or affected by the 5-HT6 receptor.

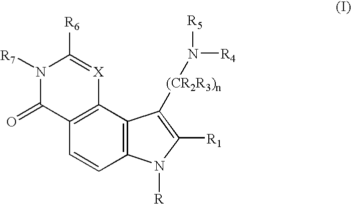

This application claims the benefit under 35 U.S.C. §119(e) to co-pending U.S. provisional application No.60/731127, filed Oct. 28, 2005, which is hereby incorporated by reference in its entirety.

### BACKGROUND OF THE INVENTION

Serotonin (5-hydroxytryptamine)(5-HT) receptors play a critical role in many physiological and behavioral functions in humans and animals. These functions are mediated through various 5-HT receptors distributed throughout the body. There are now approximately fifteen different human 5-HT receptor subtypes that have been cloned, many with well-defined roles in humans. The 5-HT6 receptor was first cloned from rat tissue in 1993 (Monsma et al., Molecular Pharmacology 1993, 43, 320-327) and subsequently from human tissue (Kohen et al., Journal of Neurochemistry 1996, 66, 47-56). The receptor is a G-protein coupled receptor (GPCR) positively coupled to adenylate cyclase (Ruat et al., Biochemical Biophysical Research Communications 1993, 193, 268-276). The receptor is found almost exclusively in the central nervous system (CNS) areas both in rat and in human. In situ hybridization studies of the 5-HT6 receptor in rat brain using mRNA indicate principal localization in the areas of 5-HT projection including striatum, nucleus accumbens, olfactory tubercle, and hippocampal formation (Ward et al., Neuroscience 1995, 64, 1105-1111).

There are many potential therapeutic uses for 5-HT6 ligands in humans based on direct effects and on indications from available scientific studies. These studies include the localization of the receptor, the affinity of ligands with known in vivo activity, and various animal studies conducted so far.

One potential therapeutic use of modulators of 5-HT6 receptor function is in the enhancement of cognition and memory in human diseases such as Alzheimer's Disease. The high levels of receptor found in important structures in the forebrain, including the caudate/putamen, hippocampus, nucleus accumbens, and cortex suggest a role for the receptor in memory and cognition, since these areas are known to play a vital role in memory (Gerard et al., Brain Research 1997, 746, 207-219). The ability of known 5-HT6 receptor ligands to enhance cholinergic transmission also supported the potential cognition use (Bentley et al., British Journal of Pharmacology 1999, 126(7), 1537-1542). Studies have found that a known 5-HT6 selective antagonist significantly increased glutamate and aspartate levels in the frontal cortex without elevating levels of noradrenaline, dopamine, or 5-HT. This selective elevation of neurochemicals known to be involved in memory and cognition strongly suggests a role for 5-HT6 ligands in cognition (Dawson et al., British Journal of Pharmacology 2000, 130(1), 23-26). Animal studies of memory and learning with a known selective 5-HT6 antagonist have found positive indications (Rogers et al., Society of Neuroscience, Abstracts 2000, 26, 680 and Foley et al., Neuropsychopharmacology 2004, 29(1), 93-100).

A related potential therapeutic use for 5-HT6 ligands is the treatment of attention deficit disorders (ADD, also known as Attention Deficit Hyperactivity Disorder or ADHD) in both children and adults. Because 5-HT6 antagonists appear to enhance the activity of the nigrostriatal dopamine pathway and because ADHD has been linked to abnormalities in the caudate (Ernst et al., Journal of Neuroscience 1998, 18(15), 5901-5907), 5-HT6 antagonists may attenuate attention deficit disorders.

Early studies examining the affinity of various CNS ligands with known therapeutic utility or a strong structural resemblance to known drugs suggests a role for 5-HT6 ligands in the treatment of schizophrenia and depression. For example, clozapine (an effective clinical antipsychotic) has high affinity for the 5-HT6 receptor subtype. Also, several clinical antidepressants have high affinity for the receptor as well and act as antagonists at this site (Branchek, et al., Annual Reviews in Pharmacology and Toxicology 2000, 40, 319-334).

Further, recent in vivo studies in rats indicate 5-HT6 modulators may be useful in the treatment of movement disorders including epilepsy (Stean et al., British Journal of Pharmacology 1999, 127 Proc. Supplement 131 P and Routledge et al., British Journal of Pharmacology 2000, 130(7), 1606-1612).

Taken together, the above studies strongly suggest that compounds which are 5-HT6 receptor modulators, i.e., ligands, may be useful for therapeutic indications including: the treatment of diseases associated with a deficit in memory, cognition, and learning such as Alzheimer's and attention deficit disorder; the treatment of personality disorders such as schizophrenia; the treatment of behavioral disorders, e.g., anxiety, depression and obsessive-compulsive disorders; the treatment of motion or motor disorders such as Parkinson's disease and epilepsy; the treatment of diseases associated with neurodegeneration such as stroke and head trauma; or withdrawal from drug addiction including addiction to nicotine, alcohol, and other substances of abuse.

Therefore, it is an object of this invention to provide compounds which are useful as therapeutic agents in the treatment of a variety of central nervous system disorders related to or affected by the 5-HT6 receptor.

It is another object of this invention to provide therapeutic methods and pharmaceutical compositions useful for the treatment of central nervous system disorders related to or affected by the 5-HT6 receptor.

It is a feature of this invention that the compounds provided may also be used to further study and elucidate the 5-HT6 receptor.

### SUMMARY OF THE INVENTION

The present invention provides a pyrroloquinolinone compound of formula I 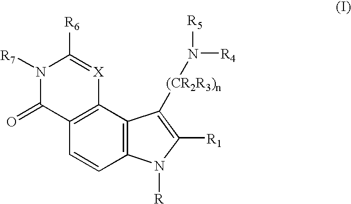
wherein
X is N or CR₈;
n is an integer of 1, 2, 3, 4, 5, or 6;
R is H, SO₂R₉ or an alkyl, cycloalkyl, alkenyl or alkynyl group each optionally substituted;
R₁ is H, halogen or an alkyl, aryl or heteroaryl group each optionally substituted;
R₂ and R₃ are each independently H or an optionally substituted alkyl group;
R₄ and R₅ are each independently H or an alkyl, alkenyl, cycloalkyl, cycloheteroalkyl, aryl or heteroaryl group each optionally substituted, or R₄ and R₅ may be taken together with the atom to which they are attached to form an optionally substituted 5- to 8-membered ring optionally containing an additional heteroatom selected from O, S or NR₇;
R₆ and R₈ are each independently H, halogen or an alkyl, alkenyl, alkynyl, alkoxy, cycloalkyl, cycloheteroalkyl, aryl or heteroaryl group each optionally substituted;
R₇ is H or an alkyl, alkenyl, alkynyl, cycloalkyl, cycloheteroalkyl, aryl, or heteroaryl group each optionally substituted; and
R₉ is an alkyl, aryl or heteroaryl group each optionally substituted; or a stereoisomer thereof or a pharmaceutically acceptable salt thereof.

The present invention also provides methods and compositions useful in the treatment of central nervous system disorders.

### DETAILED DESCRIPTION OF THE INVENTION

The 5-hydroxytryptamine-6 (5-HT6) receptor has been identified by molecular cloning. Its ability to bind a wide range of therapeutic compounds used in psychiatry, coupled with its intriguing distribution in the brain has stimulated significant interest in new compounds which are capable of interacting with or affecting said receptor. Significant efforts are being made to understand the possible role of the 5-HT6 receptor in psychiatry, cognitive dysfunction, motor function and control, memory, mood and the like. To that end, compounds which demonstrate a binding affinity for the 5-HT6 receptor are earnestly sought both as an aid in the study of the 5-HT6 receptor and as potential therapeutic agents in the treatment of central nervous system disorders, for example, see Reavill et al., Current Opinion in Investigational Drugs 2001, 2(1), 104-109.

Surprisingly, it has now been found that a pyrroloquinolinone compound of formula I demonstrates 5-HT6 affinity along with significant sub-type selectivity. Advantageously, said formula I compounds are effective therapeutic agents for the treatment of central nervous system (CNS) disorders associated with or affected by the 5-HT6 receptor. Accordingly, the present invention provides a pyrroloquinolinone compound of formula I 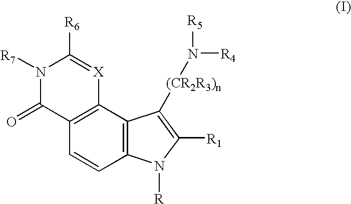
wherein
X is N or CR₈;
n is an integer of 1, 2, 3, 4, 5, or 6;
R₁ is H, SO₂R₉ or an alkyl, cycloalkyl, alkenyl or alkynyl group each optionally substituted;
R₁ is H, halogen or an alkyl, aryl or heteroaryl group each optionally substituted;
R₂ and R₃ are each independently H or an optionally substituted alkyl group;
R₄ and R₅ are each independently H or an alkyl, alkenyl, cycloalkyl, cycloheteroalkyl, aryl or heteroaryl group each optionally substituted, or R₄ and R₅ may be taken together with the atom to which they are attached to form an optionally substituted 5- to 8-membered ring optionally containing an additional heteroatom selected from O, S or NR₇;
R₆ and R₈ are each independently H, halogen or an alkyl, alkenyl, alkynyl, alkoxy, cycloalkyl, cycloheteroalkyl, aryl or heteroaryl group each optionally substituted;
R₇ is H or an alkyl, alkenyl, alkynyl, cycloalkyl, cycloheteroalkyl, aryl, or heteroaryl group each optionally substituted; and
R₉ is an alkyl, aryl or heteroaryl group each optionally substituted; or a stereoisomer thereof or a pharmaceutically acceptable salt thereof.

As used in the specification and claims, the term halogen designates F, Cl, Br or I and the term cycloheteroalkyl designates a five- to seven-membered cycloalkyl ring system containing 1 or 2 heteroatoms, which may be the same or different, selected from N, O or S and optionally containing one double bond. Exemplary of the cycloheteroalkyl ring systems included in the term as designated herein are the following rings wherein Y is NR′, O or S; and R′ is H or an optional substituent as described hereinbelow:

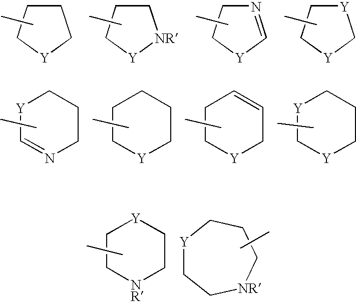

Similarly, as used in the specification and claims, the term heteroaryl designates a five- to ten-membered aromatic ring system containing 1, 2 or 3 heteroatoms, which may be the same or different, selected from N, O or S. Such heteroaryl ring systems include pyridinyl, pyrrolyl, azolyl, oxazolyl, thiazolyl, imidazolyl, furyl, thienyl, quinolinyl, isoquinolinyl, indolyl, indazolyl, azaindazolyl, benzothienyl, benzofuranyl, benzisoxazolyl or the like. The term aryl designates a carbocyclic aromatic ring system, e.g. of 6 to 14 carbon atoms such as phenyl, naphthyl, anthracenyl or the like.

As used herein, the term “alkyl” as a group or part of a group eg alkoxy, alkylamino, alkylcarbonyl (=alkanoyl), includes both (C₁-C₁₂) straight chain or branched-chain (unless defined otherwise) monovalent saturated hydrocarbon moiety. Examples of saturated hydrocarbon alkyl moieties include, but are not limited to, chemical groups of 1-6 carbon atoms such as methyl, ethyl, n-propyl, isopropyl, n-butyl, tert-butyl, isobutyl, sec-butyl; higher homologs such as n-pentyl, n-hexyl, and the like. Specifically included within the definition of “alkyl” are those alkyl groups that are optionally substituted. Suitable alkyl substitutions include, but are not limited to, CN, OH, halogen, phenyl, carbamoyl, carbonyl, alkoxy or aryloxy.

As used herein the term “haloalkyl” designates a CₙH₂ₙ₊₁, group having from one to 2n+1 halogen atoms which may be the same or different. Examples of haloalkyl groups include CF₃, CH₂Cl, C₂H₃BrCl, C₃H₅F₂, or the like.

The term “alkenyl”, as used herein, refers to either a (C₂-C₁₀) straight chain or branched-chain monovalent hydrocarbon moiety containing at least one double bond. Such hydrocarbon alkenyl moieties may be mono or polyunsaturated, and may exist in the E or Z configurations. The compounds of this invention are meant to include all possible E and Z configurations. Examples of mono or polyunsaturated hydrocarbon alkenyl moieties include, but are not limited to, chemical groups such as vinyl, 2-propenyl, isopropenyl, crotyl, 2-isopentenyl, butadienyl, 2-(butadienyl), 2,4-pentadienyl, 3-(1,4-pentadienyl), and higher homologs, isomers, or the like.

The term “alkynyl”, as used herein, refers to either a (C₂-C₁₀) straight chain or branched-chain monovalent hydrocarbon moiety containing at least one triple bond.

The term “cycloalkyl”, as used herein, refers to a monocyclic, bicyclic, tricyclic, fused, bridged, or spiro monovalent saturated hydrocarbon moiety of 3-10 carbon atoms, unless otherwise specified. Any suitable ring position of the cycloalkyl moiety may be covalently linked to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and homologs, isomers, or the like.

In the specification and claims, when the terms alkyl, alkenyl, alkynyl, cycloalkyl, cycloheteroalkyl, aryl or heteroaryl are designated as being optionally substituted, the substituent groups which are optionally present may be one or more of those customarily employed in the development of pharmaceutical compounds, or the modification of such compounds, to influence their structure/activity, persistence, absorption, stability or other beneficial property. Specific examples of optional substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylaminocarbonyl, cycloalkyl, aryl, phenyl, phenoxy, benzyl, benzyloxy, heteroaryl, indolyl, heterocyclyl, (e.g., 5-10 membered heteroaryl or heterocycloalkyl moieties containing 1-3 ring heteroatoms selected from O, S and N) or cycloalkyl groups, preferably halogen atoms or lower alkyl or lower alkoxy groups where ‘lower’ denotes 1-6 carbon atoms. Typically, 0-3 substituents may be present. When any of the foregoing substituents represents or contains an alkyl substituent group, this may be linear or branched and may contain up to 12, preferably up to 6, more preferably up to 4 carbon atoms.

Pharmaceutically acceptable salts may be any acid addition salt formed by a compound of formula I and a pharmaceutically acceptable acid such as phosphoric, sulfuric, hydrochloric, hydrobromic, citric, maleic, malonic, mandelic, succinic, fumaric, acetic, lactic, nitric, sulfonic, p-toluene sulfonic, tartaric, malic, methane sulfonic acid or the like.

Compounds of the invention include esters, carbamates or other conventional prodrug forms, which in general, are functional derivatives of the compounds of the invention and which are readily converted to the inventive active moiety in vivo. Correspondingly, the method of the invention embraces the treatment of the various conditions described hereinabove with a compound of formula I or with a compound which is not specifically disclosed but which, upon administration, converts to a compound of formula I in vivo. Also included are metabolites of the compounds of the present invention defined as active species produced upon introduction of these compounds into a biological system.

Compounds of the invention may exist as one or more stereoisomers. The various stereoisomers include enantiomers, diastereomers, atropisomers and geometric isomers. One skilled in the art will appreciate that one stereoisomer may be more active or may exhibit beneficial effects when enriched relative to the other stereoisomer(s) or when separated from the other stereoisomer(s). Additionally, the skilled artisan knows how to separate, enrich or selectively prepare said stereoisomers. Accordingly, the present invention comprises compounds of formula I, the stereoisomers thereof and the pharmaceutically acceptable salts thereof. The compounds of the invention may be present as a mixture of stereoisomers, individual stereoisomers, or as an optically active or enantiomerically pure form.

Preferred compounds of the invention are those compounds of formula I wherein n is 2. Also preferred are those compounds of formula I wherein R is H. Another group of preferred formula I compounds is those compounds wherein R₇ is H or an optionally substituted alkyl group.

More preferred compounds of the invention are those compounds of formula I wherein n is 2 and R₁ is H. Another group of more preferred compounds of the invention is those compounds of formula I wherein n is 2 and R₂ and R₃ are H. A further group of more preferred formula I compounds is those compounds wherein n is 2; R₂ and R₃ are H; and R₄ and R₅ are each independently H or alkyl.

Preferred compounds of the invention include:
1-(2-benzylaminoethyl)-6,7-dihydro-1H-pyrrolo[3,2-f]isoquinolin-6-one;
1-(2-aminoethyl)-3H,7H-pyrrolo[3,2-f]isoquinolin-6-one;
1-(2-aminoethyl)-2-bromo-3H,7H-pyrrolo[3,2-f]isoquinolin-6-one;
1-(2-aminoethyl)-7-methyl-3H,7H-pyrrolo[3,2-f]isoquinolin-6-one;
1-(2-aminoethyl)-7-benzyl-3H,7H-pyrrolo[3,2-f]isoquinolin-6-one;
1-(2-aminoethyl)-7-cyclohexylmethyl-3H,7H-pyrrolo[3,2-f]isoquinolin-6-one;
1-(2-aminoethyl)-7-thien-3-ylmethyl-3H,7H-pyrrolo[3,2-f]isoquinolin-6-one;
1-(2-aminoethyl)-7-(furan-2-ylmethyl)-3H,7H-pyrrolo[3,2-f]isoquinolin-6-one;
1-(2-methylaminoethyl)-3H,7H-pyrrolo[3,2-f]isoquinolin-6-one;
9-(2-aminoethyl)-3-benzyl-3H,7H-pyrrolo[2,3-h]quinazolin-4-one;
9-(2-aminoethyl)-3-methyl-3H,7H-pyrrolo[2,3-h]quinazolin-4-one;
9-(2-aminoethyl)-3-benzyl-3H,7H-pyrrolo[2,3-h]quinazolin-4-one;
9-(2-aminoethyl)-3H,7H-pyrrolo[2,3-h]quinazolin-4-one;
9-(2-aminoethyl)-2-(3-pyridinyl)-3H,7H-pyrrolo[2,3-h]quinazolin-4-one;
9-(2-aminoethyl)-2,3-dimethyl-3H,7H-pyrrolo[2,3-h]quinazolin-4-one;
a stereoisomer thereof; or a pharmaceutically acceptable salt thereof.

Compounds of formula I may be prepared using conventional synthetic methods and, if required, standard separation or isolation techniques. For example, compounds of formula I wherein R₄ and R₅ are H (Ia) may be prepared by reacting an isoquinolinone of formula II with a dioxolan derivative of formula III via a Fisher indole synthesis to obtain the desired compound of formula Ia. The formula Ia free amine may be reacted with an appropriate halide, R₄-Hal, in the presence of a base such as NaH to give a compound of formula I wherein R₅ is H and R₄ is other than H (Ib). Similarly, the formula Ib compound may then be reacted with another halide, R₅-Hal, in the presence of a base, to give the compound of formula I wherein R₄ and R₅ are other than H (Ic). The reactions are shown in flow diagram I wherein Hal is Cl, Br or I.

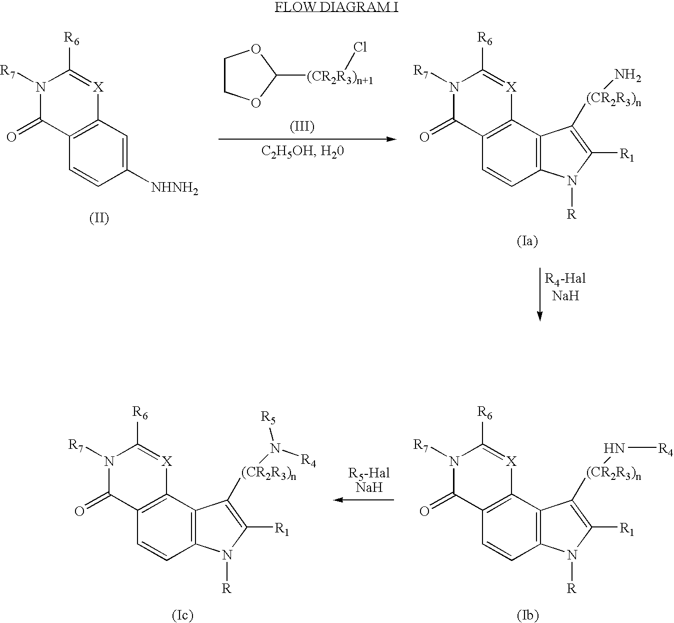

Alternatively, compounds of formula Ib may be obtained using a two-step reaction sequence involving, the reaction of la with a carboxylic acid derivative to provide the corresponding amide and subsequent reduction of said amide with a suitable reducing agent, such as LiAlH₄, to give the desired amine of formula Ib. This process may be repeated with an alternative carboxylic acid derivative with subsequent reduction to provide the fully substituted amine derivative of formula Ic.

A method useful for preparing libraries of compounds of formula Ib or Ic is the reaction of formula Ia with a suitable aldehyde derivative to form the corresponding imine and reducing said imine with a suitable reducing agent such as NaBH₄, or for ease of automation, polymer supported borohydride, to give a compound of formula Ib in high yield.

Isoquinolinones or quinazolinones of formula II are known and are commercially available or may be prepared, e.g., by the methods described by Izumi et al., J. Heterocyclic Chem. 1990, 27,1419, and Hegedus et al., J. Org. Chem. 1977, 42,1329. For example, 4-fluoro-2-ethenyl substituted benzamides of formula IV can be intramolecularly cyclized to the corresponding isoquinolinone or quinazolinone by the combined action of palladium(II)chloride and copper(I)chloride to give the fluoroisoquinolinone or fluoroquinazolinoneof formula V and reacting said formula V compound with hydrazine to give the desired compound of formula II. The reaction is shown in flow diagram II.

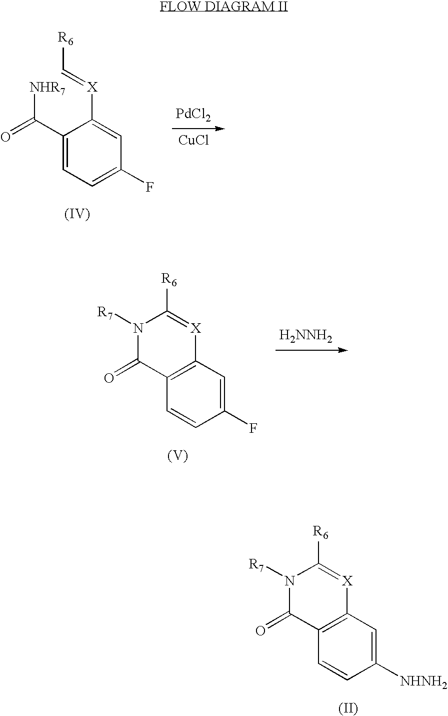

Compounds of formula V wherein X is CH may also be prepared by the method described by Heck et al. in J. Org. Chem., 1977, 42, 3903 and J. Org. Chem., 1978, 43, 2454. Compounds of formula V wherein X is N (Va) may be prepared by the cyclocondensation reaction of anthranilic acid derivatives, i.e. Rewcastle et al., J. Med. Chem. 1996, 39(4), 918-928, Hudson et al. (WO 96/09294), Houghten et al., (WO 98/11438), Fantin et al., J. Org. Chem. 1993, 58(3), 741-743, or Bhattacharya et al. (WO 97/28118, WO 97/28132 and WO 97/28134). Compounds of formula Va may also be prepared by the reaction of 2-amino-4-fluorobenzoic acid with a formamidine acetate derivative of formula VI, followed by the reaction of the formula VI compound with an electrophile, R₇-Hal, in the presence of a base to give the desired compound of formula Va. The reaction is shown in flow diagram III wherein Hal is Cl, Br or I.

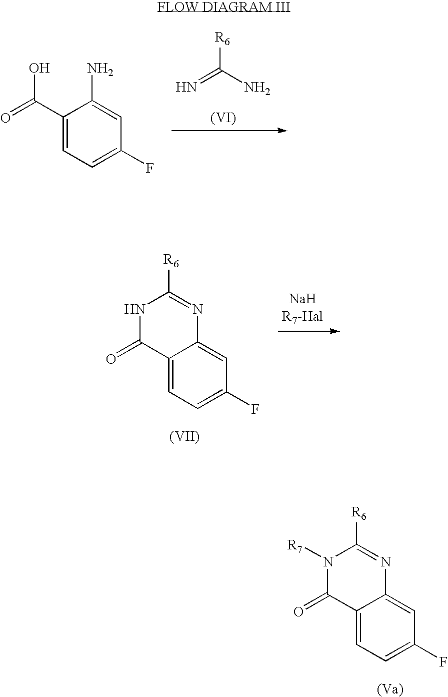

Compounds of formula V are then converted, via reaction with hydrazine as shown in flow diagram II, to compounds of formula II and the formula II compounds are then used to prepare compounds of formula I as shown in flow diagram I hereinabove.

Advantageously, the formula I compounds of the invention are useful for the treatment of CNS disorders relating to or affected by the 5-HT6 receptor including mood, personality, behavioral, psychiatric, cognitive, neurodegenerative, or the like disorders, for example, Alzheimer's disease, Parkinson's disease, attention deficit disorder, anxiety, epilepsy, depression, obsessive-compulsive disorder, sleep disorders, neurodegenerative disorders (such as head trauma or stroke), feeding disorders (such as anorexia or bulimia), schizophrenia, memory loss, disorders associated with withdrawal from drug or nicotine abuse, or the like or certain gastrointestinal disorders such as irritable bowel syndrome. Accordingly, the present invention provides a method for the treatment of a disorder of the central nervous system related to or affected by the 5-HT6 receptor in a patient in need thereof which comprises providing said patient a therapeutically effective amount of a compound of formula I as described hereinabove. The compounds may be provided by oral or parenteral administration or in any common manner known to be an effective administration of a therapeutic agent to a patient in need thereof.

The term “providing” as used herein with respect to providing a compound or substance embraced by the invention, designates either directly administering such a compound or substance, or administering a prodrug, derivative or analog which forms an equivalent amount of the compound or substance within the body. The therapeutically effective amount provided in the treatment of a specific CNS disorder may vary according to the specific condition(s) being treated, the size, age and response pattern of the patient, the severity of the disorder, the judgment of the attending physician or the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, the compounds of the invention are provided by administering the compound or a precursor thereof in a solid or liquid form, either neat or in combination with one or more conventional pharmaceutical carriers or excipients. Accordingly, the present invention provides a pharmaceutical composition which comprises a pharmaceutically acceptable carrier and an effective amount of a compound of formula I as described hereinabove.

Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid which is in admixture with a finely divided compound of formula I. In tablets, the formula I compound may be mixed with a carrier having the necessary compression properties in suitable proportions and compacted in the shape and size desired. Said powders and tablets may contain up to 99% by weight of the formula I compound. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any pharmaceutically acceptable liquid carrier suitable for preparing solutions, suspensions, emulsions, syrups and elixirs may be employed in the composition of the invention. Compounds of formula I may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

Compositions of the invention which are sterile solutions or suspensions are suitable for intramuscular, intraperitoneal or subcutaneous injection. Sterile solutions may also be administered intravenously. Inventive compositions suitable for oral administration may be in either liquid or solid composition form.

For a more clear understanding, and in order to illustrate the invention more clearly, specific examples thereof are set forth hereinbelow. The following examples are merely illustrative and are not to be understood as limiting the scope and underlying principles of the invention in any way.

Unless otherwise stated, all parts are parts by weight. The term NMR designates nuclear magnetic resonance. The terms THF, DMF and EtOAc designate tetrahydrofuran, dimethyl formamide and ethyl acetate, respectively.

### EXAMPLE 1

### 4-Fluorocinnamyl azide

An acetone solution (25 ml) of 4-fluorocinnamic acid (2.0 g, 12.0 mmol) and triethylamine (1.27 g, 12.6 mmol) was cooled to −20° C. under nitrogen and the stirred mixture was treated with ethyl chloroformate (1.76 g, 16.2 mmol). After two hours, an aqueous solution of sodium azide (1.09 g, 16.8 mmol in 5 mL) was added, and the reaction mixture was stirred for a further two hours. The mixture was filtered and concentrated to dryness to provide the acylazide as a white crystalline solid (2.2 g, 95% yield).

Elemental Analysis for: C₉H₆FN₃O Calculated: C, 56.55; H, 3.16; N, 21.98 Found: C, 56.65; H, 3.19; N, 22.01

### EXAMPLE 2

### 6-Fluoro-1-isoquinolinone

A solution of 4-fluorocinnamylazide from Example 1 (10.0 g, 52.6 mmol) in diphenyl ether (90 ml), containing a crystal of iodine, was heated at 320° C. for fifteen minutes. The mixture was cooled to ambient temperature, diluted with hexane (900 ml) and the precipitated product (6.1 g, 71% yield) was collected by filtration. The product was crystallized from acetone to afford a white solid.

Elemental Analysis for: C₉H₆FNO Calculated: C, 66.26; H, 3.71; N, 8.59 Found: C, 66.45; H, 3.92; N, 8.66

### EXAMPLE 3

### 6-Hydrazino-1-isoquinolinone

A solution of 6-fluoro-1-isoquinolinone (11.5 g, 70.5 mmol) from Example 2 and hydrazine (56.4 g, 1.76 mol) was refluxed in dioxane (175 ml) under nitrogen for 19 hours. The mixture was concentrated under vacuo, 200 ml of water added thereto, and the product collected by filtration. An ethanolic solution (500 ml) of the product was treated with 50 ml of 2N HCl, and the solution was filtered and concentrated in vacuo to afford the required product as a light yellow colored solid (12.8 g, 86% yield, mp 270° C.).

Elemental Analysis for: C₉H₉N₃O 1.0 HCl Calculated: C, 51.07; H, 4.76; N, 19.85 Found: C, 50.41; H, 4.91; N, 19.33

### EXAMPLE 4

### 1-(2-Aminoethyl)-3H,7H-pyrrolo[3,2-f]isoquinolin-6-one

A solution of 6-hydrazino-1-isoquinolinone (1.0 g, 4.73 mmol) from Example 3 and 2-(3-chloropropyl)-1,3 dioxolan (1.32 g, 8.9 mmol) in degassed ethanol (80 ml)/water (15 ml) was refluxed under nitrogen for one hour. The reaction mixture was concentrated in vacuo, and the product was purified by flash silica gel chromatography to afford an amber colored solid (0.45 g, 42% yield). Treatment of the product with ethereal HCl and crystallization from ethanol-ether gave the mono hydrochloride salt as a light amber colored solid (mp >280° C.).

Elemental Analysis for: C₁₃H₁₃N₃O, 1.0 HCl 0.3 H₂O Calculated: C, 58.02; H, 5.47; N, 15.61 Found: C, 57.92; H, 5.38; N, 15.20

### EXAMPLE 5

### 1-(2-Aminoethyl)-2-bromo-3H,7H-pyrrolo[3,2-f]isoquinolin-6-one

Bromine (1.27 mmol) was added to a stirred solution of 1-(2-aminoethyl)-3H,7H-pyrrolo[3,2-f]isoquinolin-6-one (0.240 g, 1.06 mmol) from Example 4 in DMF (2 ml). After three hours, the solution was concentrated to dryness, and the product was purified by crystallization from ethereal ethanol. Two further recrystallizations from the same solvent system provided the titled compound as a white solid (0.114 g, 27% yield, mp 252° C., decomposes).

Elemental Analysis for: C₁₃H₁₂BrN₃O 1.0 HBr Calculated: C, 40.34; H, 3.39; N, 10.89 Found: C, 40.58; H, 3.54; N, 10.46

### EXAMPLE 6

### 1-(2-N-Benzoylaminoethyl)-3H,7H-pyrrolo[3,2-f]isoquinolin-6-one

A solution of benzoyl chloride (0.928 g, 6.6 mmol) in methylene chloride (10 ml) was added dropwise to a stirred aqueous solution of 1-(2-aminoethyl)-3H,7H-pyrrolo[3,2-f]isoquinolin-6-one (0.75 g, 3.3 mmol in 10 ml) from Example 4 containing potassium carbonate (0.684 g, 4.95 mmol). After one hour, the mixture was concentrated under vacuum, and the product was purified by flash silica gel chromatography (eluting with 5% MeOH/CH₂Cl₂) to afford the required product as a light brown solid.

### EXAMPLE 7A

### 1-(2-Benzylaminoethyl)-6,7-dihydro-1H-pyrrolo[3,2-f]isoquinolin-6-one

A solution of the amide (0.47 g, 1.4 mmol) from Example 6 in THF (5 ml) was treated with an excess of borane THF (1M, 20 mmol) at room temperature under nitrogen, and the mixture was stirred for 30 hours. The reaction was terminated by the addition of HCl, and the product isolated by flash silica gel chromatography (20% MeOH/5% NH₄OH/75% CH₂Cl₂) to afford a clear solid. Treatment with ethanolic HCl afforded the salt of the titled compound as a white solid. (mp 210° C.).

Elemental Analysis for: C₂₀H₁₉N₃O 1.0 HCl Calculated: C, 67.89; H, 5.70; N, 11.87 Found: C,,67.95; H, 5.67; N, 11.98

### EXAMPLE 7B

### Library Procedure

### 1-(2-Benzylaminoethyl)-6,7-dihydro-1H-pyrrolo[3,2-f]isoquinolin-6-one

Molecular sieves (100 mg), benzaldehyde (100 μL), and 500 μL of a 0.1 M solution of the HCl salt of 1-(2-aminoethyl)-3H,7H-pyrrolo[3,2-f]isoquinolin-6-one (from Example 4) in anhydrous methanol (containing two equivalents of triethylamine) was shaken for 24 hours. Borohydride resin (57 mg) was added to the reaction well, and the mixture shaken for 16 hours at room temperature. Methanol (1 ml) was added, the mixture vortexed, and the top solution removed and concentrated. The product was dissolved in DMSO (1.6 ml), and the product purity confirmed by HPLC and MS analysis.

C₂₀H₁₉N₃O (MW 317.39)

### EXAMPLES 8-91

Using essentially the same procedure described in Example 7B and employing 1-(2-aminoethyl)-3H,7H-pyrrolo[3,2-f]isoquinolin-6-one as substrate and a suitable aldehyde, the compounds shown in Table I were obtained and identified by HPLC and mass spectral analyses.

TABLE I

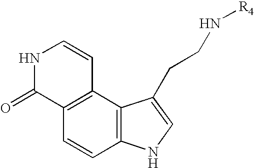

| Ex. | | |
|---|---|---|
| No. | R4 | Mass |
| 8 | naphth-2-ylmethyl | 367.45 |
| 9 | 4-phenoxybenzyl | 409.48 |
| 10 | ethyl | 255.31 |
| 11 | 2-chlorobenzyl | 351.83 |
| 12 | furfur-2-ylmethyl | 307.35 |
| 13 | 4-dimethylaminobenzyl | 360.45 |
| 14 | thiophen-2-ylmethyl | 323.41 |
| 15 | 4-methylbenzyl | 331.41 |
| 16 | 4-chlorobenzyl | 351.83 |
| 17 | 4-cyanobenzyl | 342.40 |
| 18 | n-pentyl | 297.40 |
| 19 | 3,4-methylenedioxybenzyl | 361.40 |
| 20 | 4-isopropylbenzyl | 359.47 |
| 21 | 5-methylfurfur-2-ylmethyl | 321.37 |
| 22 | 4-hydroxybenzyl | 333.38 |
| 23 | 4-methoxybenzyl | 347.41 |
| 24 | 2-methoxybenzyl | 347.41 |
| 25 | 3-fluoro-4-methoxybenzyl | 365.40 |
| 26 | 3,5-bis(trifluoromethyl)benzyl | 453.38 |
| 27 | 2,6-difluorobenzyl | 353.37 |
| 28 | 2-fluorobenzyl | 335.38 |
| 29 | 2-trifluoromethylbenzyl | 385.38 |
| 30 | 3-trifluoromethylbenzyl | 385.38 |
| 31 | 4-trifluoromethylbenzyl | 385.38 |
| 32 | 3-fluorobenzyl | 335.38 |
| 33 | 4-fluorobenzyl | 335.38 |
| 34 | indol-3-ylmethyl | 356.42 |
| 35 | furfur-3-ylmethyl | 307.35 |
| 36 | thiophen-3-ylmethyl | 323.41 |
| 37 | pyridin-3-ylmethyl | 318.37 |
| 38 | 2-methylbenzyl | 331.41 |
| 39 | 3-chlorobenzyl | 351.83 |
| 40 | 3-methylbutyl | 297.40 |
| 41 | 3-methoxybenzyl | 347.41 |
| 42 | 4-trifluoromethoxybenzyl | 401.38 |
| 43 | pyridin-4-ylmethyl | 318.37 |
| 44 | 2,4-dichlorobenzyl | 386.28 |
| 45 | pyridin-2-ylmethyl | 318.37 |
| 46 | 6-methyl-pyridin-2-ylmethyl | 332.40 |
| 47 | 4-bromobenzyl | 396.28 |
| 48 | 1-methylpyrrol-2-ylmethyl | 320.39 |
| 49 | 2,4-difluorobenzyl | 353.37 |
| 50 | 4-carbomethoxybenzyl | 375.42 |
| 51 | cyclohexylmethyl | 323.43 |
| 52 | 4-phenylbenzyl | 393.48 |
| 53 | 4-thiomethylbenzyl | 363.48 |
| 54 | 4-ethylbenzyl | 345.44 |
| 55 | quinolin-2-ylmethyl | 368.43 |
| 56 | 4-n-propyloxybenzyl | 375.47 |
| 57 | (S)-(−)-3,7-dimethyloct-6-enyl | 365.51 |
| 58 | 3,4-dichlorobenzyl | 386.28 |
| 59 | 2,4-dimethylbenzyl | 345.44 |
| 60 | 1,4-benzodioxan-6-ylmethyl | 375.42 |
| 61 | 3,5-difluorobenzyl | 353.37 |
| 62 | 3,4-difluorobenzyl | 353.37 |
| 63 | 2-chloro-4-hydroxybenzyl | 367.83 |
| 64 | 2-fluoro-3-trifluoromethylbenzyl | 403.37 |
| 65 | 3-trifluoromethoxybenzyl | 401.38 |
| 66 | 3-bromo-4-methoxybenzyl | 426.31 |
| 67 | 3-methylbenzyl | 331.41 |
| 68 | 3-bromobenzyl | 396.28 |
| 69 | 3,5-dimethoxybenzyl | 377.44 |
| 70 | 2-chloro-4-fluorobenzyl | 369.82 |
| 71 | 5-chloro-thiophen-2-ylmethyl | 357.86 |
| 72 | 2-bromobenzyl | 396.28 |
| 73 | benzofuran-2-ylmethyl | 357.41 |
| 74 | 5-bromo-furan-2-ylmethyl | 386.24 |
| 75 | 4-diethylaminobenzyl | 388.51 |
| 76 | 6-chloro-3,4-methylenedioxybenzyl | 396.84 |
| 77 | 3-bromo-4-fluorobenzyl | 414.27 |
| 78 | 3-chloro-4-fluorobenzyl | 369.82 |
| 79 | 3,5-dichlorobenzyl | 386.28 |
| 80 | 4-n-hexyloxybenzyl | 417.55 |
| 81 | trans-4-stilbene-2-yl-methyl | 419.52 |
| 82 | (R)-(+)-3,7-dimethyloct-6-enyl | 365.51 |
| 83 | 2-carboethoxy-cycloprop-1-ylmethyl | 353.42 |
| 84 | 2,3-dihydrobenzo[b]furan-5-ylmethyl | 359.42 |
| 85 | 5-bromothiophen-2-ylmethyl | 402.31 |
| 86 | 3-(3,4-dichlorophenoxy)benzyl | 478.37 |
| 87 | 3-nitrobenzyl | 362.38 |
| 88 | 3,4,5-trihydroxy-n-pentyl | 345.39 |
| 89 | 3-cyanobenzyl | 342.40 |
| 90 | 4(5)-imidazolylmethyl | 307.35 |
| 91 | 3-hydroxybenzyl | 333.38 |

### EXAMPLE 92

### 2-N-methyl-6-fluoro-1-isoquinolinone

A stirred solution of 6-fluoro-1-isoquinolinone (3.1 g, 19.0 mmol from Example 2) and sodium hydride (0.684 g, 28.5 mmol) in DMF (40 ml) was treated with methyl iodide (4.05 g, 28.5 mmol) at −30° C. After stirring under nitrogen at ambient temperature for 30 minutes, 50 ml of 1N aqueous HCl was added thereto, and the product was extracted into CH₂C1₂ (3×50 ml). The combined organic layers were washed with 50 ml of water, dried over anhydrous sodium sulfate, filtered and concentrated in vacuo to give a solid. This was crystallized from hexane/CH₂C1₂ to afford the titled product as a white solid (3.0 g, 89% yield).

Elemental Analysis for: C₁₀H₈FNO Calculated: C, 7.79; H, 4.55; N, 7.91 Found: C, 67.95; H, 4.67; N, 7.98

### EXAMPLE 93

### 2-N-methyl-6-hydrazino-1-isoquinolinone

A stirred solution of 2-N-methyl-6-fluoro-1-isoquinolinone (3.0 g, 16.9 mmol from Example 92) and hydrazine (5 g, 0.156 mmol) was refluxed in 50 ml of dioxane under nitrogen for 19 hours. The mixture was concentrated under vacuo, 50 ml of water was added thereto and the product collected by filtration. An ethanolic solution of the product (100 ml) was treated with 2N HCl (10 ml), filtered and concentrated in vacuo to afford the required product as a light yellow colored solid (3.1 g, 81% yield).

Elemental Analysis for: C₁₀H₁₁N₃O 1.0 HCl Calculated: C, 53.22; H, 5.36; N, 18.62 Found: C, 53.51; H, 5.45; N, 18.69

### EXAMPLE 94

### 1-(2-aminoethyl-7-methyl-3H,7H-pyrrolo[3,2-f]isoquinolin-6-one

A solution of 2-N-methyl-6-hydrazino-1-isoquinolinone (1.0 g, 4.4 mmol, from Example 93) was reacted in a Fisher indole reaction with 2-(3-chloropropyl)-1,3-dioxolan (0.89 g, 5.94 mmol) according to the process outlined in Example 4. After refluxing for 3.5 hours, the product was isolated in the manner described in Example 4 and purified by flash silica gel chromatography (20% MeOH, 5% NH₄0H in CH₂Cl₂) to afford a light brown oil (0.584 g, 55% yield). Treatment with ethanolic HCl gave the required product as its mono hydrochloride salt which was crystallized twice from EtOH/Et₂O to afford the product as a light tan colored solid (mp 295-298° C.).

Elemental Analysis for: C₁₄H₁₅N₃O 1.0 HCl 0.6H₂O Calculated: C, 58.27; H, 6.01; N, 14.56 Found: C, 58.11; H, 5.99; N, 14.43

### EXAMPLE 95

### 7-methyl-1-(2-phenethylaminoethyl)-3H,7H-pyrrolo[3,2-f]isoquinolin-6-one

A solution of phenacetyl chloride (0.58 g, 3.8 mmol) in methylene chloride (10 ml) was added dropwise to a stirred aqueous solution of 1-(2-aminoethyl)-7-methyl-3H,7H-pyrrolo[3,2-f]isoquinolin-6-one (0.60 g, 2.5 mmol in 10 ml) from Example 94 containing triethylamine (0.51 g, 5 mmol). After one hour, the mixture was concentrated under vacuum, and the product was purified by flash silica gel chromatography (eluting with 3% MeOH/EtOAc) to afford the required product as a cream colored solid (0.56 g, 63% yield). A solution of the amide (1.53 mmol) in THF (5 ml) was treated with an excess of borane-THF (1M, 9 mmol) at room temperature under nitrogen, and the mixture was stirred for 30 hours. The reaction was terminated by the addition of HCl, and the product was isolated by flash silica gel chromatography (10% MeOH/5% NH₄OH/85% CH₂Cl₂) to afford a light brown colored solid (0.24 g, 45% yield). Treatment with ethanolic HCl afforded the salt of the titled compound as an off white colored solid (mp 205-208° C.).

Elemental Analysis for: C₂₂H₂₃N₃O 1.0 HCl Calculated: C, 69.19; H, 6.33; N, 11.00 Found: C, 69.25; H, 6.47; N, 11.08

### EXAMPLE 96

### 7-methyl-1-(2-benzylaminoethyl)-3H,7H-pyrrolo[3.2-f]isoquinolin-6-one

A solution of benzoic anhydride (1.7 g, 7.9 mmol) in DMF (10 ml) was added dropwise to a stirred solution of 1-(2-aminoethyl)-7-methyl-3H,7H-pyrrolo[3,2-f]isoquinolin-6-one (1.2 g, 4.97 mmol in 10 ml DMF) from Example 94 containing triethylamine (0.80 g, 7.9 mmol). After one hour, the mixture was concentrated under vacuum and the product purified by flash silica gel chromatography (eluting with 5% MeOH/CH₂Cl₂) to afford the required product as an off white colored solid (0.95 g, 55% yield). A solution of the amide (2.61 mmol) in THF (30 ml) was treated with an excess of borane-THF (1M, 10.4 mmol) at room temperature under nitrogen, and the mixture was refluxed for two hours. The reaction was terminated by the addition of concentrated HCl, and the product isolated by chromatography (10% MeOH/5% NH₄OH/85% CH₂Cl₂, on A1203) to afford a tan colored solid (21% yield). Treatment with ethanolic HCl afforded the salt of the titled compound as a white solid (mp 186-192° C.).

Elemental Analysis for: C₂₁H₂₁N₃O 1.0 HCl Calculated: C, 68.56; H, 6.03; N, 11.42 Found: C, 68.75; H, 6.17; N, 11.58

### EXAMPLE 97

### 1-(2-dimethylaminoethyl)-7-methyl-3H,7H-pyrrolo[3,2-]isoquinolin-6-one

A solution of 1-(2-aminoethyl)-3H,7H-pyrrolo[3,2-f]isoquinolin-6-one (0.2 g, 0.88 mmol from Example 4) in DMF (2 mL) was treated with sodium hydroxide (2.5 N, 0.2 mmol) and methyl iodide (2.1 mmol), and the mixture was stirred at room temperature for 3 days. The product was purified by flash silica gel chromatography (50% MeOH, 5% NH₄OH in CH₂Cl₂) to afford the titled compound which was treated with ethereal HCl to afford the required salt as a white solid (0.26 mmol, 30% yield, mp 180° C.).

Elemental Analysis for: C₁₆H₁₉N₃O 1.0 HCl Calculated: C, 62.84; H, 6.59; N, 13.74 Found: C, 62.85; H, 6.67; N,13.87

### EXAMPLES 98-181

Using essentially the same procedure described in Example 7B and employing 1-(2-aminoethyl)-7-methyl-3H,7H-pyrrolo[3,2-f]isoquinolin-6-one as substrate and a suitable aldehyde. The compounds shown in Table II were obtained and identified by HPLC and mass spectral analyses.

TABLE II

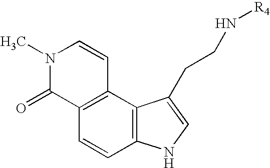

| Ex. | | |
|---|---|---|
| No. | R4 | Mass |
| 98 | naphth-2-ylmethyl | 381.47 |
| 99 | 4-phenoxybenzyl | 423.51 |
| 100 | ethyl | 269.34 |
| 101 | 2-chlorobenzyl | 365.86 |
| 102 | furfur-2-ylmethyl | 321.37 |
| 103 | 4-dimethylaminobenzyl | 374.48 |
| 104 | thiophen-2-ylmethyl | 337.44 |
| 105 | 4-methylbenzyl | 345.44 |
| 106 | 4-chlorobenzyl | 365.86 |
| 107 | 4-cyanobenzyl | 356.42 |
| 108 | n-pentyl | 311.42 |
| 109 | 3,4-methylenedioxybenzyl | 375.42 |
| 110 | 4-isopropylbenzyl | 373.49 |
| 111 | 5-methylfurfur-2-ylmethyl | 335.40 |
| 112 | 4-hydroxybenzyl | 347.41 |
| 113 | 4-methoxybenzyl | 361.44 |
| 114 | 2-methoxybenzyl | 361.44 |
| 115 | 3-fluoro-4-methoxybenzyl | 379.43 |
| 116 | 3,5-bis(trifluoromethyl)benzyl | 467.41 |
| 117 | 2,6-difluorobenzyl | 367.39 |
| 118 | 2-fluorobenzyl | 349.40 |
| 119 | 2-trifluoromethylbenzyl | 399.41 |
| 120 | 3-trifluoromethylbenzyl | 399.41 |
| 121 | 4-trifluoromethylbenzyl | 399.41 |
| 122 | 3-fluorobenzyl | 349.40 |
| 123 | 4-fluorobenzyl | 349.40 |
| 124 | indol-3-ylmethyl | 370.45 |
| 125 | furfur-3-ylmethyl | 321.37 |
| 126 | thiophen-3-ylmethyl | 337.44 |
| 127 | pyridin-3-ylmethyl | 332.40 |
| 128 | 2-methylbenzyl | 345.44 |
| 129 | 3-chlorobenzyl | 365.86 |
| 130 | 3-methylbutyl | 311.42 |
| 131 | 3-methoxybenzyl | 361.44 |
| 132 | 4-trifluoromethoxybenzyl | 415.41 |
| 133 | pyridin-4-ylmethyl | 332.40 |
| 134 | 2,4-dichlorobenzyl | 400.30 |
| 135 | pyridin-2-ylmethyl | 332.40 |
| 136 | 6-methyl-pyridin-2-ylmethyl | 346.43 |
| 137 | 4-bromobenzyl | 410.31 |
| 138 | 1-methylpyrrol-2-ylmethyl | 334.42 |
| 139 | 2,4-difluorobenzyl | 367.39 |
| 140 | 4-carbomethoxybenzyl | 389.45 |
| 141 | cyclohexylmethyl | 337.46 |
| 142 | 4-phenylbenzyl | 407.51 |
| 143 | 4-thiomethylbenzyl | 377.51 |
| 144 | 4-ethylbenzyl | 359.47 |
| 145 | quinolin-2-ylmethyl | 382.46 |
| 146 | 4-n-propyloxybenzyl | 389.49 |
| 147 | (S)-(−)-3,7-dimethyloct-6-enyl | 379.54 |
| 148 | 3,4-dichlorobenzyl | 400.30 |
| 149 | 2,4-dimethylbenzyl | 359.47 |
| 150 | 1,4-benzodioxan-6-ylmethyl | 389.45 |
| 151 | 3,5-difluorobenzyl | 367.39 |
| 152 | 3,4-difluorobenzyl | 367.39 |
| 153 | 2-chloro-4-hydroxybenzyl | 381.86 |
| 154 | 2-fluoro-3-trifluoromethylbenzyl | 417.40 |
| 155 | 3-trifluoromethoxybenzyl | 415.41 |
| 156 | 3-bromo-4-methoxybenzyl | 440.33 |
| 157 | 3-methylbenzyl | 345.44 |
| 158 | 3-bromobenzyl | 410.31 |
| 159 | 3,5-dimethoxybenzyl | 391.46 |
| 160 | 2-chloro-4-fluorobenzyl | 383.85 |
| 161 | 5-chloro-thiophen-2-ylmethyl | 371.89 |
| 162 | 2-bromobenzyl | 410.31 |
| 163 | benzofuran-2-ylmethyl | 371.43 |
| 164 | 5-bromo-furan-2-ylmethyl | 400.27 |
| 165 | 4-diethylaminobenzyl | 402.53 |
| 166 | 6-chloro-3,4-methylenedioxybenzyl | 409.87 |
| 167 | 3-bromo-4-fluorobenzyl | 428.30 |
| 168 | 3-chloro-4-fluorobenzyl | 383.85 |
| 169 | 3,5-dichlorobenzyl | 400.30 |
| 170 | 4-n-hexyloxybenzyl | 431.57 |
| 171 | trans-4-stilbene-2-yl-methyl | 433.55 |
| 172 | (R)-(+)-3,7-dimethyloct-6-enyl | 379.54 |
| 173 | 2-carboethoxy-cyclopropyl-1-ylmethyl | 367.44 |
| 174 | 2,3-dihydrobenzo[b]furan-5-ylmethyl | 373.45 |
| 175 | 5-bromothiophen-2-ylmethyl | 416.34 |
| 176 | 3-(3,4-dichlorophenoxy)benzyl | 492.40 |
| 177 | 3-nitrobenzyl | 376.41 |
| 178 | 3,4,5-trihydroxy-n-pentyl | 359.42 |
| 179 | 3-cyanobenzyl | 356.42 |
| 180 | 4(5)-imidazolylmethyl | 321.38 |
| 181 | 3-hydroxybenzyl | 347.41 |

### EXAMPLE 182

### 1-2-aminoethyl)-7-benzyl-3H,7H-pyrrolo[3,2-f]isoquinolin-6-one

A solution of 2-N-benzyl-6-hydrazino-1-isoquinolinone (0.5 g, 1.66 mmol, prepared from benzyl bromide in 66% yield according to Examples 92 and 93 was reacted in a Fisher indole reaction with 2-(3-chloropropyl)-1,3-dioxolan (0.34 g, 2.24 mmol) according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product (0.162 g, 27% yield) as its mono hydrochloride salt as an off white colored solid (mp 266-268° C.).

Elemental Analysis for: C₂₀H₁₉N₃O 1.0 HCl 1.3H₂O calculated: C 63.67; H, 6.04; N, 11.14 Found: C, 63.59; H, 6.04; N, 11.01

### EXAMPLE 183

### 1-2-aminoethyl)-7-benzyl-3-methyl-3H,7H-pyrrolo[3,2-f]isoquinolin-6-one

A solution of 1-(2-aminoethyl)-7-benzyl-3H,7H-pyrrolo[3,2-f]isoquinolin-6-one (0.25 g, 0.79 mmol) from Example 182 and methyl iodide (0.135 g, 0.95 mmol) in DMF (2 ml) was treated with sodium hydride (0.022 g, 0.91 mmol), and the resulting mixture was stirred at 0° C. under nitrogen for one hour. Water (10 ml) was added and the product extracted with ethyl acetate (2×15 ml). The combined organics were washed with 15 ml of brine, separated and dried over anhydrous sodium sulfate. Filtration and concentration in vacuo gave a light brown colored solid (0.21 g). The product was purified by flash silica gel chromatography (10% MeOH/5% NH4OH in CH₂Cl₂) to afford a white solid. Treatment with ethanolic HCl gave the titled compound as its mono hydrochloride salt as a white solid (0.138 g, 48% yield, mp 172-176° C.).

Elemental Analysis for: C₂₁H₂₁N₃O 1.0 HCl 1H₂O Calculated: C, 65.36; H, 6.27; N, 10.89 Found: C, 65.16; H, 6.04; N, 10.52

### EXAMPLES 184-267

Using essentially the same procedure described in Example 7B and employing 1-(2-aminoethyl)-7-benzyl-3H,7H-pyrrolo[3,2-f]isoquinolin-6-one as substrate and a suitable aldehyde, the compounds shown in Table III were obtained and identified by HPLC and mass spectral analyses.

TABLE III

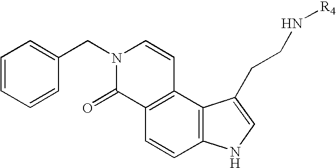

| Ex. | | |
|---|---|---|
| No. | R4 | Mass |
| 184 | naphth-2-ylmethyl | 457.57 |
| 185 | 4-phenoxybenzyl | 499.61 |
| 186 | ethyl | 345.44 |
| 187 | 2-chlorobenzyl | 441.95 |
| 188 | furfur-2-ylmethyl | 397.47 |
| 189 | 4-dimethylaminobenzyl | 450.58 |
| 190 | thiophen-2-ylmethyl | 413.54 |
| 191 | 4-methylbenzyl | 421.54 |
| 192 | 4-chlorobenzyl | 441.95 |
| 193 | 4-cyanobenzyl | 432.59 |
| 194 | n-pentyl | 387.52 |
| 195 | 3,4-methylenedioxybenzyl | 451.52 |
| 196 | 4-isopropylbenzyl | 449.59 |
| 197 | 5-methylfurfur-2-ylmethyl | 441.5 |
| 198 | 4-hydroxybenzyl | 423.51 |
| 199 | 4-methoxybenzyl | 437.54 |
| 200 | 2-methoxybenzyl | 437.54 |
| 201 | 3-fluoro-4-methoxybenzyl | 455.53 |
| 202 | 3,5-bis(trifluoromethyl)benzyl | 543.51 |
| 203 | 2,6-difluorobenzyl | 443.49 |
| 204 | 2-fluorobenzyl | 425.50 |
| 205 | 2-trifluoromethylbenzyl | 475.51 |
| 206 | 3-trifluoromethylbenzyl | 475.51 |
| 207 | 4-trifluoromethylbenzyl | 475.51 |
| 208 | 3-fluorobenzyl | 425.50 |
| 209 | 4-fluorobenzyl | 425.50 |
| 210 | indol-3-ylmethyl | 446.55 |
| 211 | furfur-3-ylmethyl | 397.47 |
| 212 | thiophen-3-ylmethyl | 413.54 |
| 213 | pyridin-3-ylmethyl | 408.50 |
| 214 | 2-methylbenzyl | 421.54 |
| 215 | 3-chlorobenzyl | 441.95 |
| 216 | 3-methylbutyl | 387.52 |
| 217 | 3-methoxybenzyl | 437.54 |
| 218 | 4-trifluoromethoxybenzyl | 491.51 |
| 219 | pyridin-4-ylmethyl | 408.50 |
| 220 | 2,4-dichlorobenzyl | 476.40 |
| 221 | pyridin-2-ylmethyl | 408.50 |
| 222 | 6-methyl-pyridin-2-ylmethyl | 422.52 |
| 223 | 4-bromobenzyl | 486.41 |
| 224 | 1-methylpyrrol-2-ylmethyl | 410.51 |
| 225 | 2,4-difluorobenzyl | 443.49 |
| 226 | 4-carbomethoxybenzyl | 465.55 |
| 227 | cyclohexylmethyl | 413.56 |
| 228 | 4-phenylbenzyl | 483.61 |
| 229 | 4-thiomethylbenzyl | 453.60 |
| 230 | 4-ethylbenzyl | 435.56 |
| 231 | quinolin-2-ylmethyl | 458.56 |
| 232 | 4-n-propyloxybenzyl | 465.59 |
| 233 | (S)-(−)-3,7-dimethyloct-6-enyl | 455.64 |
| 234 | 3,4-dichlorobenzyl | 476.40 |
| 235 | 2,4-dimethylbenzyl | 435.56 |
| 236 | 1,4-benzodioxan-6-ylmethyl | 465.55 |
| 237 | 3,5-difluorobenzyl | 443.49 |
| 238 | 3,4-difluorobenzyl | 443.49 |
| 239 | 2-chloro-4-hydroxybenzyl | 457.95 |
| 240 | 2-fluoro-3-trifluoromethylbenzyl | 493.50 |
| 241 | 3-trifluoromethoxybenzyl | 491.51 |
| 242 | 3-bromo-4-methoxybenzyl | 516.43 |
| 243 | 3-methylbenzyl | 421.54 |
| 244 | 3-bromobenzyl | 486.41 |
| 245 | 3,5-dimethoxybenzyl | 467.56 |
| 246 | 2-chloro-4-fluorobenzyl | 459.95 |
| 247 | 5-chloro-thiophen-2-ylmethyl | 447.98 |
| 248 | 2-bromobenzyl | 486.41 |
| 249 | benzofuran-2-ylmethyl | 447.53 |
| 250 | 5-bromo-furan-2-ylmethyl | 476.37 |
| 251 | 4-diethylaminobenzyl | 478.63 |
| 252 | 6-chloro-3,4-methylenedioxybenzyl | 485.96 |
| 253 | 3-bromo-4-fluorobenzyl | 504.40 |
| 254 | 3-chloro-4-fluorobenzyl | 459.95 |
| 255 | 3,5-dichlorobenzyl | 476.40 |
| 256 | 4-n-hexyloxybenzyl | 507.67 |
| 257 | trans-4-stilbene-2-yl-methyl | 509.65 |
| 258 | (R)-(+)-3,7-dimethyloct-6-enyl | 455.64 |
| 259 | 2-carboethoxy-cycloprop-1-ylmethyl | 443.54 |
| 260 | 2,3-dihydrobenzo[b]furan-5-ylmethyl | 449.55 |
| 261 | 5-bromothiophen-2-ylmethyl | 492.43 |
| 262 | 3-(3,4-dichlorophenoxy)benzyl | 568.50 |
| 263 | 3-nitrobenzyl | 452.51 |
| 264 | 3,4,5-trihydroxy-n-pentyl | 435.52 |
| 265 | 3-cyanobenzyl | 432.52 |
| 266 | 4(5)-imidazolylmethyl | 397.47 |
| 267 | 3-hydroxybenzyl | 423.51 |

### EXAMPLE 268

### 1-(3-aminopropyl)-3H,7H-pyrrolo[3,2-f]isoquinolin-6-one

A solution of 6-hydrazino-1-isoquinolinone (1.0 g, 4.73 mmol) from Example 3 and 2-(4-chlorobutyl)-1,3-dioxolan (1.05 g, 6.39 mmol) in degassed ethanol (80 ml)/water (16 ml) was refluxed under nitrogen for five hours. The reaction mixture was concentrated in vacuo and the product purified by flash silica gel chromatography (20% MeOH/5% NH₄OH in CH₂Cl₂) to afford a light amber colored solid (0.08 g, 7% yield). Treatment of the product with ethereal HCl and crystallization from ethanol ether gave the mono hydrochloride salt of the titled compound as a light tan colored solid (mp 225-230° C.).

Elemental Analysis for: C₁₄H₁₅N₃O 1.0 HCl Calculated: C, 60.54; H, 5.81; N, 15.13 Found: C, 60.65; H, 5.87; N, 15.19

### EXAMPLE 269

### 1-(2-aminoethyl)-7-cyclohexylmethyl-3H,7H-pyrrolo[3,2-]isoquinolin-6-one

A solution of 2-N-cyclohexylmethyl-6-hydrazino-1-isoquinolinone (1.0 g, 3.2 mmol, prepared from cyclohexylmethylbromide in 69% yield according to Example 92 and Example 93) was reacted in a Fisher indole reaction with 2-(3-chloropropyl)-1,3-dioxolan (0.651 g, 4.32 mmol) in a mixture of degassed ethanol (80 ml) and water (20 ml) according to the process outlined in Example 4. After refluxing for three hours, the product was isolated as reported above and purified by flash silica gel chromatography (20% MeOH/5% NH₄OH in CH₂Cl₂) to afford a viscous brown oil (0.8 g). Treatment with ethanolic HCl gave the required product as its mono hydrochloride salt which was crystallized twice from EtOH/Et₂O to afford the product as a light brown colored solid (0.33 g, 28% yield, mp 220-224° C.).

Elemental Analysis for: C₂₀H₂₅N₃O 1.0 HCl 1.5H₂O Calculated: C, 62.09; H, 7.55; N, 10.86 Found: C, 62.32; H, 7.27; N, 10.56

### EXAMPLE 270

### 1-(2-aminoethyl)-7-phenylethyl-3H,7H-pyrrolo[3,2-f]isoquinolin-6-one

A solution of 2-N-phenylethyl-6-hydrazino-1-isoquinolinone (1.0 g, 3.2 mmol, prepared from 2-phenylethylbromide in 72% yield in the manner described in Example 92 and Example 93) was reacted in a Fisher indole reaction with 2-(3-chloropropyl)-1,3-dioxolan (0.65 g, 4.3 mmol) in a mixture of degassed ethanol (80 ml) and water (20 ml) according to the process outlined in Example 4. After refluxing for three hours, the product was isolated as reported above and purified by flash silica gel chromatography (20% MeOH/5% NH₄OH in CH₂Cl₂) to afford a light brown colored solid (0.37 g). Treatment with ethanolic HCl gave the required product as its mono hydrochloride salt which was crystallized twice from EtOH/Et₂O to afford the product as a white solid (0.36 g, 31% yield, mp 162-166° C.).

Elemental Analysis for: C₂₁H₂₁N₃O 1.0 HCl 1.75H₂O Calculated: C, 63.15; H, 6.43; N, 10.52 Found: C, 63.47; H, 6.14; N, 10.52

### EXAMPLE 271

### 1-(2-aminoethyl)-7-naphthalen-2-ylmethyl-3H,7H-pyrrolo[3,2-f]isoquinolin-6-one

A solution of 2-N-naphthalen-2-ylmethyl-6-hydrazino-1-isoquinolinone (2.25 g, 6.40 mmol, prepared from naphthalen-2-ylmethylbromide in 59% yield according to Example 92 and Example 93) was reacted in a Fisher indole reaction with 2-(3-chloropropyl)-1,3-dioxolan (1.3 g, 8.64 mmol) in degassed ethanol (130 ml)/water (90 ml) according to the process outlined in Example 4. After refluxing for three hours, the product was isolated as reported above and purified by flash silica gel chromatography (20% MeOH/5% NH₄OH in CH₂Cl₂) to afford an off white colored solid (0.43 g, 18% yield). Treatment with ethanolic HCl gave the titled product as its mono hydrochloride salt which was crystallized from EtOH/Et₂O to afford an off white colored solid (0.43 g, mp 288-292° C.).

Elemental Analysis for: C₂₄H₂₁N₃₀ 1.0 HCl 0.25 H₂O Calculated: C, 70.58; H, 5.55; N, 10.29 Found: C, 70.64; H, 5.62; N, 10.17

### EXAMPLE 272

### 1-(2-aminoethyl)-7-heptyl-3H,7H-pyrrolo[3,2-f]isoquinolin-6-one

A solution of 2-N-heptyl-6-hydrazino-1-isoquinolinone (0.95 g, 3.07 mmol, prepared from heptylbromide in 72% yield according to Example 92 and Example 93) was reacted in a Fisher indole reaction with 2-(3-chloropropyl)-1,3-dioxolan (0.623 g, 4.14 mmol) in degassed ethanol (80 ml)/water (20 ml) according to the process outlined in Example 4. After refluxing for 3.5 hours, the product was isolated as reported above and purified by flash silica gel chromatography (20% MeOH/5% NH₄OH in CH₂Cl₂) to afford an off white colored solid (0.33 g, 33% yield). Treatment with ethanolic HCl gave the titled product as its mono hydrochloride salt which was crystallized from EtOH/Et₂O to afford a white solid (0.33 g, mp 239-241° C.).

Elemental Analysis for: C₂₀H₂₇N₃₀ 1.0 HCl Calculated: C, 66.37; H, 7.80; N, 11.61 Found: C, 65.95; H, 7.51; N, 11.64

### EXAMPLE 273

### 1-(2-aminoethyl)-7-(34-dichlorobenzyl)-3H,7H-pyrrolo[3.2-f]isoquinolin-6-one

A solution of 2-N-(3,4-dichlorobenzyl)-6-hydrazino-1-isoquinolinone (1.30 g, 3.51 mmol, prepared from 3,4-dichlorobenzyl bromide in 79% yield according to Example 92 and Example 93) was reacted in a Fisher indole reaction with 2-(3-chloropropyl)-1,3-dioxolan (0.80 g, 5.31 mmol) in a mixture of degassed ethanol (100ml) and water (25 ml) according to the process outlined in Example 4. After refluxing for three hours, the product was isolated as reported above and purified by flash silica gel chromatography (20% MeOH/5% NH₄OH in CH₂Cl₂) to afford a light yellow colored solid (0.395 g). Treatment with ethanolic HCl gave the titled product as its mono hydrochloride salt which was crystallized from EtOH/Et₂O to afford a white solid (0.37 g, 27% yield, mp 188-190° C.).

Elemental Analysis for: C₂₀H₁₇N_{3 OCl}₂ 1.0 HCl 1H₂O Calculated: C, 54.50; H, 4.57; N, 9.53 Found: C, 54.76; H, 4.36; N, 9.31

### EXAMPLE 274

### 1-(2-aminoethyl)-7-thiophen-3-ylmethyl-3H,7H-pyrrolo[3,2-f]isoquinolin-6-one

A solution of 2-N-(thiophen-3-ylmethyl)-6-hydrazino-1-isoquinolinone (0.5 g, 1.62 mmol, prepared from thiophen-3-ylmethyl bromide in 64% yield according to Example 92 and Example 93) was reacted in a Fisher indole reaction with 2-(3-chloropropyl)-1,3-dioxolan (0.33 g, 2.19 mmol) in a mixture of degassed ethanol (40 ml) and water (8 ml) according to the process outlined in Example 4. After refluxing for four hours, the product was isolated as reported above and purified by flash silica gel chromatography (20% MeOH/5% NH₄OH in CH₂Cl₂) to afford a yellow colored solid (0.17 g, 32% yield). Treatment with ethanolic HCl gave the titled product as its mono hydrochloride salt which was crystallized from EtOH/Et₂O to afford a light yellow colored solid (0.17 g, mp 260-264° C.).

Elemental Analysis for: CB₈H₁₇N₃OS 1.0 HCl 1.1H₂O Calculated: C, 56.94; H, 5.36; N, 11.07 Found: C, 56.63; H, 5.30; N, 10.66

### EXAMPLE 275

### 3-methyl-6-fluoro-1-isoquinolinone

A suspension of 4-fluoro-2-isopropenylbenzamide (2.50 g, 14 mmol), palladium (II) chloride (0.248 g, 1.4 mmol) and copper (I) chloride (1.386 g, 14 mmol) in DME (25 ml) was stirred under nitrogen at 60° C. for 24 hours. The mixture was cooled, filtered and concentrated in vacuo. The product was purified by crystallization twice from acetone to afford a white solid (1.3 g, 52% yield).

Elemental Analysis for: C₁₀H₈FNO Calculated: C, 67.79; H, 4.55; N, 7.91 Found: C, 68.01; H, 4.67; N, 8.10

### EXAMPLE 276

### 3-methyl-6-hydrazino-1-isoquinolinone

The titled compound was obtained as a light yellow colored solid in 84% yield from 3-methyl-6-fluoro-1-isoquinolinone using the procedure described in Example 3.

Elemental Analysis for: C₁₀H₁₁N₃O 1.0 HCl Calculated: C, 53.22; H, 5.36; N, 18.62 Found: C, 53.54; H, 5.46; N, 18.80

### EXAMPLE 277

### 1-(2-aminoethyl)-8-methyl-3H,7H-pyrrolo[3.2-f]isoquinolin-6-one

A solution of 3-methyl-6-hydrazino-1-isoquinolinone (1.0 g, 5.2 mmol) and 2-(3-chloropropyl)-1,3-dioxolan (1.32 g, 8.9 mmol) in a mixture of degassed ethanol (80 ml) and water (15 ml) was refluxed under nitrogen for four hours. The reaction mixture was concentrated in vacuo and the product purified by flash silica gel chromatography to afford an amber colored solid (0.71 g, 56% yield). Treatment of the product with ethereal HCl and crystallization from ethanol ether gave the mono hydrochloride salt as a light amber colored solid (m.p. >200° C.).

Elemental Analysis for: C₁₄H₁₅N₃O 1.0 HCl Calculated: C, 60.54; H, 5.81; N, 15.13 Found: C, 60.45; H, 5.88; N, 15.20

### EXAMPLE 278

### 1-(2-aminoethyl)-2-(4-chlorophenyl)-3H,7H-pyrrolo[3,2-f]isoquinolin-6-one

A solution of 6-hydrazino-1-isoquinolinone (1.0 g, 4.73 mmol) from Example 3 and 4,4′-dichlorobutyrophenone (1.39 g, 6.4 mmol, 1.35 equivalents) in degassed ethanol (50 ml)/water (50 ml) was refluxed under nitrogen for six hours. The reaction mixture was concentrated in vacuo, and the product was purified by flash silica gel chromatography to afford a yellow colored solid (0.21 g, 13% yield). Treatment of the product with methanolic HCl and crystallization from ethanol ether gave the hydrochloride salt as a light tan colored solid (mp >300° C.).

Elemental Analysis for: C₁₉H₁₆ClN₃O 2.0 HCl Calculated: C, 55.56; H, 4.42; N, 10.23 Found: C, 55.63; H, 4.56; N, 10.20

### EXAMPLE 279

### 1-(2-aminoethyl)-9-chloro-3H,7H-pyrrolo[3.2-f]isoquinolin-6-one

A solution of 4-chloro-6-hydrazino-1-isoquinolinone (0.51 g, 2.07 mmol) and 2-(3-chloropropyl)-1,3-dioxolan (0.39 g, 2.59 mmol, 1.25 equivalents) in degassed ethanol (40 ml)/water (8 ml) was refluxed under nitrogen for four hours. The reaction mixture was concentrated in vacuo and the product purified by flash silica gel chromatography to afford a light brown colored solid (0.16 g, 29% yield). Treatment of the product with methanolic HCl and crystallization from ethanol ether gave the mono hydrochloride salt as a light tan colored solid (mp 239-243° C.).

Elemental Analysis for: C₁₃H₁₂ClN₃O 1.0 HCl Calculated: C, 52.37; H, 4.39; N, 14.39 Found: C, 52.48; H, 4.56; N, 14.55

### EXAMPLE 280

### 1-(2-aminoethyl)-7-ethyl-3H,7H-pyrrolo[3,2-f]isoquinolin-6-one

A solution of 2-N-ethyl-6-hydrazino-1-isoquinolinone (0.45 g, 1.88 mmol, prepared from iodoethane by the method described in Example 92 and Example 93) in degassed ethanol (40 ml)/water (8 ml) was reacted in a Fisher indole reaction with 2-(3-chloropropyl)-1,3-dioxolan (0.354 g, 2.35 mmol, 1.25 equivalents) according to the process outlined in Example 4. After refluxing for 4 hours, the product was isolated in the manner described in Example 4 and purified by chromatography (neutral alumina, type III, eluting with 4% MeOH in CH₂Cl₂) to afford a light yellow oil (0.105 g, 22% yield). Treatment with ethanolic HCl gave the required product as its mono hydrochloride salt which was crystallized from EtOH/Et₂O to afford the product as a white solid (mp 224-227° C.).

Elemental Analysis for: C₁₅H₁₇N₃O 1.0 HCl Calculated: C, 61.75; H, 6.22; N, 14.40 Found: C, 61.95; H, 6.49; N, 14.43

### EXAMPLES 281-288

Using essentially the same procedure described in Example 7B and employing 1-(2-aminoethyl)-7-ethyl-3H,7H-pyrrolo[3,2-f]isoquinolin-6-one as substrate and a suitable aldehyde, the compounds shown in Table IV were obtained and identified by HPLC and mass spectral analyses.

TABLE IV

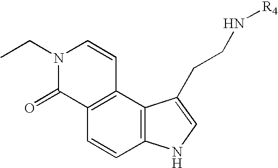

| Ex. No. | R4 | Mass |
|---|---|---|
| 281 | 3-nitrobenzyl | 390.44 |
| 282 | 3,4,5-trihydroxy-n-pentyl | 373.45 |
| 283 | 3-chlorobenzyl | 379.88 |
| 284 | 3-methoxybenzyl | 375.47 |
| 285 | pyridin-4-ylmethyl | 346.43 |
| 286 | 3-cyanobenzyl | 370.45 |
| 287 | 4(5)-imidazolylmethyl | 335.40 |
| 288 | 3-hydroxybenzyl | 361.44 |

### EXAMPLE 289

### 1-(2-aminoethyl)-7-(tetrahydropyran-2-ylmethyl)-3H,7H-pyrrolo[3,2-f]isoquinolin-6-one

A solution of 2-N-(tetrahydropyran-2-ylmethyl)-6-hydrazinoisoquinolinone (0.70 g, 2.26 mmol, prepared from tetrahydropyran-2-ylmethylbromide by the method described in Example 92 and Example 93) in degassed ethanol (55 ml)/water (10 ml) was reacted in a Fisher indole reaction with 2-(3-chloropropyl)-1,3-dioxolan (0.426 g, 2.83 mmol, 1.25 equivalents) according to the process outlined in Example 4. After refluxing for 4 hours, the product was isolated in the manner described in Example 4, and purified by chromatography (neutral alumina, type III, eluting with 5% MeOH in CH₂Cl₂) to afford a yellow colored solid (0.251 g, 34% yield). Treatment with ethanolic HCl gave the required product as its mono hydrochloride salt which was crystallized from EtOH/Et₂O to afford the product as a white solid (mp 260-262° C.). Elemental Analysis for: C₁₉H₂₃N₃O₂ 1.0 HCl Calculated: C, 63.06; H, 6.68; N, 11.61 Found: C, 63.25; H, 6.69; N, 11.63

### EXAMPLE 290

### 1-(2-aminoethyl)-7-(2-methoxyethyl)-3H,7H-pyrrolo[3.2-f]isoquinolin-6-one

A solution of 2-N-(2-methoxyethyl)-6-hydrazinoisoquinolinone (1.37 g, 5.87 mmol, prepared from 1-bromo-2-methoxyethane by the method described in Example 92 and Example 93) in degassed ethanol (100 ml)/water (16 ml) was reacted in a Fisher indole reaction with 2-(3-chloropropyl)-1,3-dioxolan (1.11 g, 7.34 mmol, 1.25 equivalents) according to the process outlined in Example 4. After refluxing for 4 hours, the product was isolated in the manner described in Example 4 and purified by chromatography (silica gel, eluting with 8% MeOH in CH₂Cl₂) to afford a yellow colored solid (0.200 g, 12% yield). Treatment with ethanolic HCl gave the required product as its mono hydrochloride salt which was crystallized from EtOH/Et₂O to afford the product as a white solid (mp 246-249° C.).

Elemental Analysis for: C₁₆H₁₉N₃O₂ 1.0 HCl Calculated: C, 59.72; H, 6.26; N, 13.06 Found: C, 59.92; H, 6.39; N, 13.23

### EXAMPLE 291

### 1-(2-aminoethyl)-7-(4-fluorobenzyl)-3H,7H-Pyrrolo[3,2-f]isoquinolin-6-one

A solution of 2-N-(4-fluorobenzyl)-6-hydrazinoisoquinolinone (0.88 g, 3.10 mmol, prepared from 4-fluorobenzylbromide by the method described in Example 92 and Example 93) in degassed ethanol (100 ml)/water (16 ml) was reacted in a Fisher indole reaction with 2-(3-chloropropyl)-1,3-dioxolan (0.46 g, 3.1 mmol) according to the process outlined in Example 4. After refluxing for 16 hours, the product was isolated in the manner described in Example 4 and purified by chromatography (silica gel, eluting with 10% MeOH inCH₂Cl₂) to afford a yellow colored solid (0.45 g). Treatment with ethanolic HCl gave the required product as its mono hydrochloride salt as a pale yellow colored solid (mp 247-249° C.).

Elemental Analysis for: C₂₀H₁₈FN₃O 1.0 HCl Calculated: C, 64.60; H, 5.15; N, 11.30 Found: C, 64.72; H, 5.39; N, 11.33

### EXAMPLES 292-376

Using essentially the same procedure described in Example 7B and employing 1-(2-aminoethyl)-7-(4-fluorobenzyl)-3H,7H-pyrrolo[3,2-f]isoquinolin-6-one as substrate and a suitable aldehyde, the compounds shown in Table V were obtained and identified by HPLC and mass spectral analyses.

TABLE V

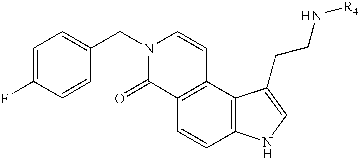

| Ex. | | |
|---|---|---|
| No. | R4 | Mass |
| 292 | naphth-2-ylmethyl | 475.56 |
| 293 | 4-phenoxybenzyl | 517.60 |
| 294 | ethyl | 363.43 |
| 295 | 2-chlorobenzyl | 459.95 |
| 296 | furfur-2-ylmethyl | 415.46 |
| 297 | 4-dimethylaminobenzyl | 468.57 |
| 298 | benzyl | 425.50 |
| 299 | thiophen-2-ylmethyl | 431.53 |
| 300 | 4-methylbenzyl | 439.53 |
| 301 | 4-chlorobenzyl | 459.95 |
| 302 | 4-cyanobenzyl | 450.51 |
| 303 | n-pentyl | 405.51 |
| 304 | 3,4-methylenedioxybenzyl | 469.51 |
| 305 | 4-isopropylbenzyl | 467.58 |
| 306 | 5-methylfurfur-2-ylmethyl | 429.49 |
| 307 | 4-hydroxybenzyl | 441.50 |
| 308 | 4-methoxybenzyl | 455.53 |
| 309 | 2-methoxybenzyl | 455.53 |
| 310 | 3-fluoro-4-methoxybenzyl | 473.52 |
| 311 | 3,5-bis(trifluoromethyl)benzyl | 561.50 |
| 312 | 2,6-difluorobenzyl | 461.48 |
| 313 | 2-fluorobenzyl | 443.49 |
| 314 | 2-trifluoromethylbenzyl | 493.50 |
| 315 | 3-trifluoromethylbenzyl | 493.50 |
| 316 | 4-trifluoromethylbenzyl | 493.50 |
| 317 | 3-fluorobenzyl | 443.49 |
| 318 | 4-fluorobenzyl | 443.49 |
| 319 | indol-3-ylmethyl | 464.54 |
| 320 | furfur-3-ylmethyl | 415.46 |
| 321 | thiophen-3-ylmethyl | 431.53 |
| 322 | pyridin-3-ylmethyl | 426.49 |
| 323 | 2-methylbenzyl | 439.53 |
| 324 | 3-chlorobenzyl | 459.95 |
| 325 | 3-methylbutyl | 405.51 |
| 326 | 3-methoxybenzyl | 455.53 |
| 327 | 4-trifluoromethoxybenzyl | 509.50 |
| 328 | pyridin-4-ylmethyl | 426.49 |
| 329 | 2,4-dichlorobenzyl | 494.39 |
| 330 | pyridin-2-ylmethyl | 426.49 |
| 331 | 6-methyl-pyridin-2-ylmethyl | 440.52 |
| 332 | 4-bromobenzyl | 504.40 |
| 333 | 1-methylpyrrol-2-ylmethyl | 428.50 |
| 334 | 2,4-difluorobenzyl | 461.48 |
| 335 | 4-carbomethoxybenzyl | 483.54 |
| 336 | cyclohexylmethyl | 431.55 |
| 337 | 4-phenylbenzyl | 401.60 |
| 338 | 4-thiomethylbenzyl | 471.59 |
| 339 | 4-ethylbenzyl | 453.55 |
| 340 | quinolin-2-ylmethyl | 476.55 |
| 341 | 4-n-propyloxybenzyl | 483.58 |
| 342 | (S)-(-)-3,7-dimethyloct-6-enyl | 473.63 |
| 343 | 3,4-dichlorobenzyl | 494.39 |
| 344 | 2,4-dimethylbenzyl | 453.55 |
| 345 | 1,4-benzodioxan-6-ylmethyl | 483.54 |
| 346 | 3,5-difluorobenzyl | 461.48 |
| 347 | 3,4-difluorobenzyl | 461.48 |
| 348 | 2-chloro-4-hydroxybenzyl | 475.94 |
| 349 | 2-fluoro-3-trifluoromethylbenzyl | 511.49 |
| 350 | 3-trifluoromethoxybenzyl | 509.50 |
| 351 | 3-bromo-4-methoxybenzyl | 534.42 |
| 352 | 3-methylbenzyl | 539.55 |
| 353 | 3-bromobenzyl | 504.40 |
| 354 | 3,5-dimethoxybenzyl | 485.55 |
| 355 | 2-chloro-4-fluorobenzyl | 477.94 |
| 356 | 5-chloro-thiophen-2-ylmethyl | 465.97 |
| 357 | 2-bromobenzyl | 504.40 |
| 358 | benzofuran-2-ylmethyl | 465.52 |
| 359 | 5-bromo-furan-2-ylmethyl | 494.36 |
| 360 | 4-diethylaminobenzyl | 496.62 |
| 361 | 6-chloro-3,4-methylenedioxybenzyl | 503.96 |
| 362 | 3-bromo-4-fluorobenzyl | 522.39 |
| 363 | 3-chloro-4-fluorobenzyl | 477.94 |
| 364 | 3,5-dichlorobenzyl | 494.39 |
| 365 | 4-n-hexyloxybenzyl | 525.66 |
| 366 | trans-4-stilbene-2-yl-methyl | 527.64 |
| 367 | (R)-(+)-3,7-dimethyloct-6-enyl | 473.63 |
| 368 | 2-carboethoxy-cycloprop-1-ylmethyl | 461.53 |
| 369 | 2,3-dihydrobenzo[b]furan-5-ylmethyl | 467.54 |
| 370 | 5-bromothiophen-2-ylmethyl | 510.42 |
| 371 | 3-(3,4-dichlorophenoxy)benzyl | 586.49 |
| 372 | 3-nitrobenzyl | 470.50 |
| 373 | 3,4,5-trihydroxy-n-pentyl | 453.51 |
| 374 | 3-cyanobenzyl | 450.51 |
| 375 | 4(5)-imidazolylmethyl | 415.47 |
| 376 | 3-hydroxybenzyl | 441.50 |

TABLE V

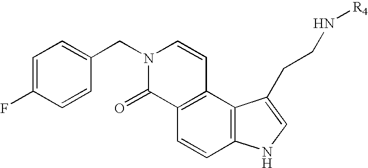

| Ex. | | |
|---|---|---|
| No. | R4 | Mass |
| 313 | 2-fluorobenzyl | 443.49 |
| 314 | 2-trifluoromethylbenzyl | 493.50 |
| 315 | 3-trifluoromethylbenzyl | 493.50 |
| 316 | 4-trifluoromethylbenzyl | 493.50 |
| 317 | 3-fluorobenzyl | 443.49 |
| 318 | 4-fluorobenzyl | 443.49 |
| 319 | indol-3-ylmethyl | 464.54 |
| 320 | furfur-3-ylmethyl | 415.46 |
| 321 | thiophen-3-ylmethyl | 431.53 |
| 322 | pyridin-3-ylmethyl | 426.49 |
| 323 | 2-methylbenzyl | 439.53 |
| 324 | 3-chlorobenzyl | 459.95 |
| 325 | 3-methylbutyl | 405.51 |
| 326 | 3-methoxybenzyl | 455.53 |
| 327 | 4-trifluoromethoxybenzyl | 509.50 |
| 328 | pyridin-4-ylmethyl | 426.49 |
| 329 | 2,4-dichlorobenzyl | 494.39 |
| 330 | pyridin-2-ylmethyl | 426.49 |
| 331 | 6-methyl-pyridin-2-ylmethyl | 440.52 |
| 332 | 4-bromobenzyl | 504.40 |
| 333 | 1-methylpyrrol-2-ylmethyl | 428.50 |
| 334 | 2,4-difluorobenzyl | 461.48 |
| 335 | 4-carbomethoxybenzyl | 483.54 |
| 336 | cyclohexylmethyl | 431.55 |
| 337 | 4-phenylbenzyl | 401.60 |
| 338 | 4-thiomethylbenzyl | 471.59 |
| 339 | 4-ethylbenzyl | 453.55 |
| 340 | quinolin-2-ylmethyl | 476.55 |
| 341 | 4-n-propyloxybenzyl | 483.58 |
| 342 | (S)-(−)-3,7-dimethyloct-6-enyl | 473.63 |
| 343 | 3,4-dichlorobenzyl | 494.39 |
| 344 | 2,4-dimethylbenzyl | 453.55 |
| 345 | 1,4-benzodioxan-6-ylmethyl | 483.54 |
| 346 | 3,5-difluorobenzyl | 461.48 |
| 347 | 3,4-difluorobenzyl | 461.48 |
| 348 | 2-chloro-4-hydroxybenzyl | 475.94 |
| 349 | 2-fluoro-3-trifluoromethylbenzyl | 511.49 |
| 350 | 3-trifluoromethoxybenzyl | 509.50 |
| 351 | 3-bromo-4-methoxybenzyl | 534.42 |
| 352 | 3-methylbenzyl | 539.55 |
| 353 | 3-bromobenzyl | 504.40 |
| 354 | 3,5-dimethoxybenzyl | 485.55 |
| 355 | 2-chloro-4-fluorobenzyl | 477.94 |
| 356 | 5-chloro-thiophen-2-ylmethyl | 465.97 |
| 357 | 2-bromobenzyl | 504.40 |
| 358 | benzofuran-2-ylmethyl | 465.52 |
| 359 | 5-bromo-furan-2-ylmethyl | 494.36 |
| 360 | 4-diethylaminobenzyl | 496.62 |
| 361 | 6-chloro-3,4-methylenedioxybenzyl | 503.96 |
| 362 | 3-bromo-4-fluorobenzyl | 522.39 |
| 363 | 3-chloro-4-fluorobenzyl | 477.94 |
| 364 | 3,5-dichlorobenzyl | 494.39 |
| 365 | 4-n-hexyloxybenzyl | 525.66 |
| 366 | trans-4-stilbene-2-yl-methyl | 527.64 |
| 367 | (R)-(+)-3,7-dimethyloct-6-enyl | 473.63 |
| 368 | 2-carboethoxy-cycloprop-1-ylmethyl | 461.53 |
| 369 | 2,3-dihydrobenzo[b]furan-5-ylmethyl | 467.54 |
| 370 | 5-bromothiophen-2-ylmethyl | 510.42 |
| 371 | 3-(3,4-dichlorophenoxy)benzyl | 586.49 |
| 372 | 3-nitrobenzyl | 470.50 |
| 373 | 3,4,5-trihydroxy-n-pentyl | 453.51 |
| 374 | 3-cyanobenzyl | 450.51 |
| 375 | 4(5)-imidazolylmethyl | 415.47 |
| 376 | 3-hydroxybenzyl | 441.50 |

### EXAMPLE 377

### 1-(2-aminoethyl)-7-(2-fluorobenzyl)-3H,7H-pyrrolo[3,2-f]isoquinolin-6-one

A solution of 2-N-(2-fluorobenzyl)-6-hydrazinoisoquinolinone (0.95 g, 3.35 mmol, prepared from 2-fluorobenzylbromide by the method described in Example 92 and Example 93) in degassed ethanol (100 ml)/water (16 ml) was reacted in a Fisher indole reaction with 2-(3-chloropropyl)-1,3-dioxolan (0.5 g, 3.35 mmol) according to the process outlined in Example 4. After refluxing for 16 hours, the product was isolated in the manner described in Example 4 and purified by chromatography (silica gel, eluting with 10% MeOH in CH₂Cl₂) to afford a yellow colored solid (0.47 g). Treatment with ethanolic HCl gave the required product as its mono hydrochloride salt as a pale yellow colored solid (mp >250° C.).

Elemental Analysis for: C₂₀H₁₈FN₃O 1.0 HCl Calculated: C, 64.60; H, 5.15; N, 11.30 Found: C, 64.79; H, 5.27; N, 11.42

### EXAMPLES 378-462

Using essentially the same procedure described in Example 7B and employing 1-(2-aminoethyl)-7-(2-fluorobenzyl)-3H,7H-pyrrolo[3,2-f]isoquinolin-6-one as substrate and a suitable aldehyde, the compounds shown in Table VI were obtained and identified by HPLC and mass spectral analyses.

TABLE VI

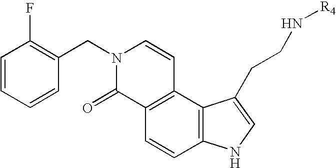

| Ex. | | |
|---|---|---|
| No. | R4 | Mass |
| 378 | naphth-2-ylmethyl | 475.56 |
| 379 | 4-phenoxybenzyl | 517.60 |
| 380 | ethyl | 363.43 |
| 381 | 2-chlorobenzyl | 459.95 |
| 382 | furfur-2-ylmethyl | 415.46 |
| 383 | 4-dimethylaminobenzyl | 468.57 |
| 384 | benzyl | 425.50 |
| 385 | thiophen-2-ylmethyl | 431.53 |
| 386 | 4-methylbenzyl | 439.53 |
| 387 | 4-chlorobenzyl | 459.95 |
| 388 | 4-cyanobenzyl | 450.51 |
| 389 | n-pentyl | 405.51 |
| 390 | 3,4-methylenedioxybenzyl | 469.51 |
| 391 | 4-isopropylbenzyl | 467.58 |
| 392 | 5-methylfurfur-2-ylmethyl | 429.49 |
| 393 | 4-hydroxybenzyl | 441.50 |
| 394 | 4-methoxybenzyl | 455.53 |
| 395 | 2-methoxybenzyl | 455.53 |
| 396 | 3-fluoro-4-methoxybenzyl | 473.52 |
| 397 | 3,5-bis(trifluoromethyl)benzyl | 561.50 |
| 398 | 2,6-difluorobenzyl | 461.48 |
| 399 | 2-fluorobenzyl | 443.49 |
| 400 | 2-trifluoromethylbenzyl | 493.50 |
| 401 | 3-trifluoromethylbenzyl | 493.50 |
| 402 | 4-trifluoromethylbenzyl | 493.50 |
| 403 | 3-fluorobenzyl | 443.49 |
| 404 | 4-fluorobenzyl | 443.49 |
| 405 | indol-3-ylmethyl | 464.54 |
| 406 | furfur-3-ylmethyl | 415.46 |
| 407 | thiophen-3-ylmethyl | 431.53 |
| 408 | pyridin-3-ylmethyl | 426.49 |
| 409 | 2-methylbenzyl | 439.53 |
| 410 | 3-chlorobenzyl | 459.95 |
| 411 | 3-methylbutyl | 405.51 |
| 412 | 3-methoxybenzyl | 455.53 |
| 413 | 4-trifluoromethoxybenzyl | 509.50 |
| 414 | pyridin-4-ylmethyl | 426.49 |
| 415 | 2,4-dichlorobenzyl | 494.39 |
| 416 | pyridin-2-ylmethyl | 426.49 |
| 417 | 6-methyl-pyridin-2-ylmethyl | 440.52 |
| 418 | 4-bromobenzyl | 504.40 |
| 419 | 1-methylpyrrol-2-ylmethyl | 428.50 |
| 420 | 2,4-difluorobenzyl | 461.48 |
| 421 | 4-carbomethoxybenzyl | 483.54 |
| 422 | cyclohexylmethyl | 431.55 |
| 423 | 4-phenylbenzyl | 401.60 |
| 424 | 4-thiomethylbenzyl | 471.59 |
| 425 | 4-ethylbenzyl | 453.55 |
| 426 | quinolin-2-ylmethyl | 476.55 |
| 427 | 4-n-propyloxybenzyl | 483.58 |
| 428 | (S)-(−)-3,7-dimethyloct-6-enyl | 473.63 |
| 429 | 3,4-dichlorobenzyl | 494.39 |
| 430 | 2,4-dimethylbenzyl | 453.55 |
| 431 | 1,4-benzodioxan-6-ylmethyl | 483.54 |
| 432 | 3,5-difluorobenzyl | 461.48 |
| 433 | 3,4-difluorobenzyl | 461.48 |
| 434 | 2-chloro-4-hydroxybenzyl | 475.94 |
| 435 | 2-fluoro-3-trifluoromethylbenzyl | 511.49 |
| 436 | 3-trifluoromethoxybenzyl | 509.50 |
| 437 | 3-bromo-4-methoxybenzyl | 534.42 |
| 438 | 3-methylbenzyl | 539.55 |
| 439 | 3-bromobenzyl | 504.40 |
| 440 | 3,5-dimethoxybenzyl | 485.55 |
| 441 | 2-chloro-4-fluorobenzyl | 477.94 |
| 442 | 5-chloro-thiophen-2-ylmethyl | 465.97 |
| 443 | 2-bromobenzyl | 504.40 |
| 444 | benzofuran-2-ylmethyl | 465.52 |
| 445 | 5-bromo-furan-2-ylmethyl | 494.36 |
| 446 | 4-diethylaminobenzyl | 496.62 |
| 447 | 6-chloro-3,4-methylenedioxybenzyl | 503.96 |
| 448 | 3-bromo-4-fluorobenzyl | 522.39 |
| 449 | 3-chloro-4-fluorobenzyl | 477.94 |
| 450 | 3,5-dichlorobenzyl | 494.39 |
| 451 | 4-n-hexyloxybenzyl | 525.66 |
| 452 | trans-4-stilbene-2-yl-methyl | 527.64 |
| 453 | (R)-(+)-3,7-dimethyloct-6-enyl | 473.63 |
| 454 | 2-carboethoxy-cycloprop-1-ylmethyl | 461.53 |
| 455 | 2,3-dihydrobenzo[b]furan-5-ylmethyl | 467.54 |
| 456 | 5-bromothiophen-2-ylmethyl | 510.42 |
| 457 | 3-(3,4-dichlorophenoxy)benzyl | 586.49 |
| 458 | 3-nitrobenzyl | 470.50 |
| 459 | 3,4,5-trihydroxy-n-pentyl | 453.51 |
| 460 | 3-cyanobenzyl | 450.51 |
| 461 | 4(5)-imidazolylmethyl | 415.47 |
| 462 | 3-hydroxybenzyl | 441.50 |

### EXAMPLE 463

### 1-(2-aminoethyl)-7-thiophen-2-ylmethyl-3H,7H-pyrrolo[3,2-f]isoquinolin-6-one

A solution of 2-N-(thiophen-2-ylmethyl)-6-hydrazino-1-isoquinolinone (prepared from 2-thienyl bromide according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid, MW=359.87; HPLC single component;identified by HNMR.

Elemental Analysis for: C₁₈H₁₇N₃OS 1.0 HCl Calculated: C, 64.07; H, 5.04; N, 11.68 Found: C, 64.09; H, 5.07; N, 11.72

### EXAMPLES 464-548

Using essentially the same procedure described in Example 7B and employing 1-(2-aminoethyl)-7-(thiophen-2-ylmethyl)-3H,7H-pyrrolo[3,2-f]isoquinolin-6-one as substrate and a suitable aldehyde, the compounds shown in Table VII were obtained and identified by HPLC and mass spectral analyses.

TABLE VII

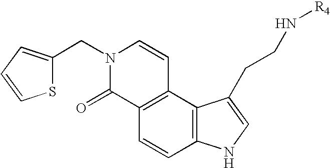

| Ex. | | |
|---|---|---|
| No. | R4 | Mass |
| 464 | naphthyl-2-ylmethyl | 463.60 |
| 465 | 4-phenoxybenzyl | 505.64 |
| 466 | ethyl | 351.47 |
| 467 | 2-chlorobenzyl | 447.98 |
| 468 | furfur-2-ylmethyl | 403.50 |
| 469 | 4-dimethylaminobenzyl | 456.61 |
| 470 | benzyl | 413.54 |
| 471 | thiophen-2-ylmethyl | 419.57 |
| 472 | 4-methylbenzyl | 427.57 |
| 473 | 4-chlorobenzyl | 447.98 |
| 474 | 4-cyanobenzyl | 438.55 |
| 475 | n-pentyl | 393.55 |
| 476 | 3,4-methylenedioxybenzyl | 457.55 |
| 477 | 4-isopropylbenzyl | 455.62 |
| 478 | 5-methylfurfur-2-ylmethyl | 417.53 |
| 479 | 4-hydroxybenzyl | 429.54 |
| 480 | 4-methoxybenzyl | 443.56 |
| 481 | 2-methoxybenzyl | 443.56 |
| 482 | 3-fluoro-4-methoxybenzyl | 461.56 |
| 483 | 3,5-bis(trifluoromethyl)benzyl | 549.53 |
| 484 | 2,6-difluorobenzyl | 449.52 |
| 485 | 2-fluorobenzyl | 431.53 |
| 486 | 2-trifluoromethylbenzyl | 481.54 |
| 487 | 3-trifluoromethylbenzyl | 481.54 |
| 488 | 4-trifluoromethylbenzyl | 481.54 |
| 489 | 3-fluorobenzyl | 431.53 |
| 490 | 4-fluorobenzyl | 431.53 |
| 491 | indol-3-ylmethyl | 452.57 |
| 492 | furfur-3-ylmethyl | 403.50 |
| 493 | thiophen-3-ylmethyl | 419.57 |
| 494 | pyridin-3-ylmethyl | 414.53 |
| 495 | 2-methylbenzyl | 427.57 |
| 496 | 3-chlorobenzyl | 447.98 |
| 497 | 3-methylbutyl | 393.55 |
| 498 | 3-methoxybenzyl | 443.56 |
| 499 | 4-trifluoromethoxybenzyl | 497.54 |
| 500 | pyridin-4-ylmethyl | 414.53 |
| 501 | 2,4-dichlorobenzyl | 482.43 |
| 502 | pyridin-2-ylmethyl | 414.53 |
| 503 | 6-methyl-pyridin-2-ylmethyl | 428.55 |
| 504 | 4-bromobenzyl | 492.43 |
| 505 | 1-methylpyrrol-2-ylmethyl | 416.54 |
| 506 | 2,4-difluorobenzyl | 449.52 |
| 507 | 4-carbomethoxybenzyl | 471.57 |
| 508 | cyclohexylmethyl | 419.59 |
| 509 | 4-phenylbenzyl | 489.64 |
| 510 | 4-thiomethylbenzyl | 459.63 |
| 511 | 4-ethylbenzyl | 441.59 |
| 512 | quinolin-2-ylmethyl | 464.59 |
| 513 | 4-n-propyloxybenzyl | 471.62 |
| 514 | (S)-(−)-3,7-dimethyloct-6-enyl | 461.67 |
| 515 | 3,4-dichlorobenzyl | 482.43 |
| 516 | 2,4-dimethylbenzyl | 441.59 |
| 517 | 1,4-benzodioxan-6-ylmethyl | 471.57 |
| 518 | 3,5-difluorobenzyl | 449.52 |
| 519 | 3,4-difluorobenzyl | 449.52 |
| 520 | 2-chloro-4-hydroxybenzyl | 463.98 |
| 521 | 2-fluoro-3-trifluoromethylbenzyl | 499.53 |
| 522 | 3-trifluoromethoxybenzyl | 497.54 |
| 523 | 3-bromo-4-methoxybenzyl | 522.46 |
| 524 | 3-methylbenzyl | 427.57 |
| 525 | 3-bromobenzyl | 492.43 |
| 526 | 3,5-dimethoxybenzyl | 473.59 |
| 527 | 2-chloro-4-fluorobenzyl | 465.97 |
| 528 | 5-chloro-thiophen-2-ylmethyl | 454.01 |
| 529 | 2-bromobenzyl | 492.43 |
| 530 | benzofuran-2-ylmethyl | 453.56 |
| 531 | 5-bromo-furan-2-ylmethyl. | 482.40 |
| 532 | 4-diethylaminobenzyl | 484.66 |
| 533 | 6-chloro-3,4-methylenedioxybenzyl | 491.99 |
| 534 | 3-bromo-4-fluorobenzyl | 510.42 |
| 535 | 3-chloro-4-fluorobenzyl | 565.97 |
| 536 | 3,5-dichlorobenzyl | 482.43 |
| 537 | 4-n-hexyloxybenzyl | 513.70 |
| 538 | trans-4-stilbene-2-yl-methyl | 515.67 |
| 539 | (R)-(+)-3,7-dimethyloct-6-enyl | 461.67 |
| 540 | 2-carboethoxy-cycloprop-1-ylmethyl | 449.57 |
| 541 | 2,3-dihydrobenzo[b]furan-5-ylmethyl | 455.58 |
| 542 | 5-bromothiophen-2-ylmethyl | 498.46 |
| 543 | 3-(3,4-dichlorophenoxy)benzyl | 574.52 |
| 544 | 3-nitrobenzyl | 458.54 |
| 545 | 3,4,5-trihydroxy-n-pentyl | 441.55 |
| 546 | 3-cyanobenzyl | 438.55 |
| 547 | 4(5)-imidazolylmethyl | 403.50 |
| 548 | 3-hydroxybenzyl | 429.54 |

### EXAMPLE 549

### 1-(2-aminoethyl)-7-cyclopropylmethyl-3H,7H-pyrrolo[3,2-f]isoquinolin-6-one

A solution of 2-N-cyclopropylmethyl-6-hydrazino-1-isoquinolinone (prepared from bromomethylcyclopropane according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=317.81; HPLC single component; 1H-NMR, 3.9 δ, 2H, d).

Elemental Analysis for: C₁₇H₁₉N₃O 1.0 HCl Calculated: C, 64.25; H, 6.34; N, 13.22 Found: C, 64.49; H, 6.47; N, 13.42

### EXAMPLES 550-634

Using essentially the same procedure described in Example 7B and employing 1-(2-aminoethyl)-7-cyclopropylmethyl-3H,7H-pyrrolo[3,2-f]isoquinolin-6-one as substrate and a suitable aldehyde, the compounds shown in Table VII were obtained and identified by HPLC and mass spectral analyses.

TABLE VIII

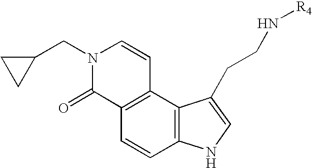

| Ex. | | |
|---|---|---|
| No. | R4 | Mass |
| 550 | naphth-2-ylmethyl | 421.54 |
| 551 | 4-phenoxybenzyl | 463.57 |
| 552 | ethyl | 309.41 |
| 553 | 2-chlorobenzyl | 405.92 |
| 554 | furfur-2-ylmethyl | 361.44 |
| 555 | 4-dimethylaminobenzyl | 414.55 |
| 556 | benzyl | 371.48 |
| 557 | thiophen-2-ylmethyl | 377.51 |
| 558 | 4-methylbenzyl | 385.50 |
| 559 | 4-chlorobenzyl | 405.92 |
| 560 | 4-cyanobenzyl | 396.49 |
| 561 | n-pentyl | 351.49 |
| 562 | 3,4-methylenedioxybenzyl | 415.49 |
| 563 | 4-isopropylbenzyl | 413.56 |
| 564 | 5-methylfurfur-2-ylmethyl | 375.47 |
| 565 | 4-hydroxybenzyl | 387.48 |
| 566 | 4-methoxybenzyl | 401.50 |
| 567 | 2-methoxybenzyl | 401.50 |
| 568 | 3-fluoro-4-methoxybenzyl | 419.49 |
| 569 | 3,5-bis(trifluoromethyl)benzyl | 507.47 |
| 570 | 2,6-difluorobenzyl | 407.46 |
| 571 | 2-fluorobenzyl | 389.47 |
| 572 | 2-trifluoromethylbenzyl | 439.48 |
| 573 | 3-trifluoromethylbenzyl | 439.48 |
| 574 | 4-trifluoromethylbenzyl | 439.48 |
| 575 | 3-fluorobenzyl | 389.47 |
| 576 | 4-fluorobenzyl | 389.47 |
| 577 | indol-3-ylmethyl | 410.51 |
| 578 | furfur-3-ylmethyl | 361.44 |
| 579 | thiophen-3-ylmethyl | 377.51 |
| 580 | pyridin-3-ylmethyl | 372.46 |
| 581 | 2-methylbenzyl | 385.50 |
| 582 | 3-chlorobenzyl | 405.92 |
| 583 | 3-methylbutyl | 351.49 |
| 584 | 3-methoxybenzyl | 401.50 |
| 585 | 4-trifluoromethoxybenzyl | 455.47 |
| 586 | pyridin-4-ylmethyl | 372.46 |
| 587 | 2,4-dichlorobenzyl | 440.37 |
| 588 | pyridine-2-ylmethyl | 372.46 |
| 589 | 6-methyl-pyridin-2-ylmethyl | 386.49 |
| 590 | 4-bromobenzyl | 450.37 |
| 591 | 1-methylpyrrol-2-ylmethyl | 374.48 |
| 592 | 2,4-difluorobenzyl | 407.46 |
| 593 | 4-carbomethoxybenzyl | 429.51 |
| 594 | cyclohexylmethyl | 377.52 |
| 595 | 4-phenylbenzyl | 447.57 |
| 596 | 4-thiomethylbenzyl | 417.57 |
| 597 | 4-ethylbenzyl | 399.53 |
| 598 | quinolin-2-ylmethyl | 422.52 |
| 599 | 4-n-propyloxybenzyl | 429.56 |
| 600 | (S)-(−)-3,7-dimethyloct-6-enyl | 419.61 |
| 601 | 3,4-dichlorobenzyl | 440.37 |
| 602 | 2,4-dimethylbenzyl | 399.53 |
| 603 | 1,4-benzodioxan-6-ylmethyl | 429.51 |
| 604 | 3,5-difluorobenzyl | 407.46 |
| 605 | 3,4-difluorobenzyl | 407.46 |
| 606 | 2-chloro-4-hydroxybenzyl | 421.92 |
| 607 | 2-fluoro-3-trifluoromethylbenzyl | 457.47 |
| 608 | 3-trifluoromethoxybenzyl | 455.47 |
| 609 | 3-bromo-4-methoxybenzyl | 480.40 |
| 610 | 3-methylbenzyl | 385.50 |
| 611 | 3-bromobenzyl | 450.37 |
| 612 | 3,5-dimethoxybenzyl | 431.53 |
| 613 | 2-chloro-4-fluorobenzyl | 423.91 |
| 614 | 5-chloro-thiophen-2-ylmethyl | 411.95 |
| 615 | 2-bromobenzyl | 450.37 |
| 616 | benzofuran-2-ylmethyl | 411.50 |
| 617 | 5-bromo-furan-2-ylmethyl | 440.33 |
| 618 | 4-diethylaminobenzyl | 442.60 |
| 619 | 6-chloro-3,4-methylenedioxybenzyl | 449.93 |
| 620 | 3-bromo-4-fluorobenzyl | 468.37 |
| 621 | 3-chloro-4-fluorobenzyl | 423.91 |
| 622 | 3,5-dichlorobenzyl | 440.37 |
| 623 | 4-n-hexyloxybenzyl | 471.64 |
| 624 | trans-4-stilbene-2-yl-methyl | 473.61 |
| 625 | (R)-(+)-3,7-dimethyloct-6-enyl | 419.61 |
| 626 | 2-carboethoxy-cycloprop-1-ylmethyl | 407.51 |
| 627 | 2,3-dihydrobenzo[b]furan-5-ylmethyl | 413.51 |
| 628 | 5-bromothiophen-2-ylmethyl | 456.40 |
| 629 | 3-(3,4-dichlorophenoxy)benzyl | 432.46 |
| 630 | 3-nitrobenzyl | 458.54 |
| 631 | 3,4,5-trihydroxy-n-pentyl | 399.49 |
| 632 | 3-cyanobenzyl | 396.49 |
| 633 | 4(5)-imidazolylmethyl | 361.44 |
| 634 | 3-hydroxybenzyl | 387.48 |

### EXAMPLE 635

### 1-(2-aminoethyl)-7-propyl-3H,7H-pyrrolo[3,2-f]isoquinolin-6-one

A solution of 2-N-propyl-6-hydrazino-1-isoquinolinone (prepared from n-propyl bromide according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=305.13; HPLC single component; 1H-NMR, 0.9 6 δ, t, 3H; 3.9 δ, 2H, m).

Elemental Analysis for: C₁₆H₁₉N₃O 1.0 HCl Calculated; C, 62.84; H, 6.59; N, 13.74 Found: C, 62.96; H, 6.66; N, 13.82

### EXAMPLES 636-720

Using essentially the same procedure described in Example 7B and employing 1-(2-aminoethyl)-7-propyl-3H,7H-pyrrolo[3,2-f]isoquinolin-6-one as substrate and a suitable aldehyde, the compounds shown in Table IX were obtained and identified by HPLC and mass spectral analyses.

TABLE IX

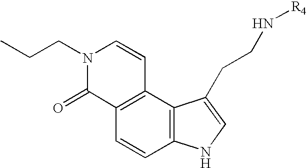

| Ex. | | |
|---|---|---|
| No. | R4 | Mass |
| 636 | naphth-2-ylmethyl | 409.53 |
| 637 | 4-phenoxybenzyl | 451.56 |
| 638 | ethyl | 297.40 |
| 639 | 2-chlorobenzyl | 393.91 |
| 640 | furfur-2-ylmethyl | 349.43 |
| 641 | 4-dimethylaminobenzyl | 402.53 |
| 642 | benzyl | 359.47 |
| 643 | thiophen-2-ylmethyl | 365.49 |
| 644 | 4-methylbenzyl | 373.49 |
| 645 | 4-chlorobenzyl | 393.91 |
| 646 | 4-cyanobenzyl | 384.48 |
| 647 | n-pentyl | 339.48 |
| 648 | 3,4-methylenedioxybenzyl | 403.48 |
| 649 | 4-isopropylbenzyl | 401.55 |
| 650 | 5-methylfurfur-2-ylmethyl | 363.45 |
| 651 | 4-hydroxybenzyl | 375.47 |
| 652 | 4-methoxybenzyl | 389.49 |
| 653 | 2-methoxybenzyl | 389.49 |
| 654 | 3-fluoro-4-methoxybenzyl | 407.48 |
| 655 | 3,5-bis(trifluoromethyl)benzyl | 495.46 |
| 656 | 2,6-difluorobenzyl | 395.42 |
| 657 | 2-fluorobenzyl | 377.46 |
| 658 | 2-trifluoromethylbenzyl | 427.46 |
| 659 | 3-trifluoromethylbenzyl | 427.46 |
| 660 | 4-trifluoromethylbenzyl | 427.46 |
| 661 | 3-fluorobenzyl | 377.46 |
| 662 | 4-fluorobenzyl | 377.46 |
| 663 | indol-3-ylmethyl | 398.50 |
| 664 | furfur-3-ylmethyl | 349.43 |
| 665 | thiophen-3-ylmethyl | 365.49 |
| 666 | pyridin-3-ylmethyl | 360.45 |
| 667 | 2-methylbenzyl | 373.49 |
| 668 | 3-chlorobenzyl | 393.91 |
| 669 | 3-methylbutyl | 339.48 |
| 670 | 3-methoxybenzyl | 389.49 |
| 671 | 4-trifluoromethoxybenzyl | 443.46 |
| 672 | pyridin-4-ylmethyl | 360.45 |
| 673 | 2,4-dichlorobenzyl | 428.36 |
| 674 | pyridin-2-ylmethyl | 360.45 |
| 675 | 6-methyl-pyridin-2-ylmethyl | 374.48 |
| 676 | 4-bromobenzyl | 438.36 |
| 677 | 1-methylpyrrol-2-ylmethyl | 362.47 |
| 678 | 2,4-difluorobenzyl | 395.45 |
| 679 | 4-carbomethoxybenzyl | 417.50 |
| 680 | cyclohexylmethyl | 365.51 |
| 681 | 4-phenylbenzyl | 435.56 |
| 682 | 4-thiomethylbenzyl | 405.56 |
| 683 | 4-ethylbenzyl | 387.52 |
| 684 | quinolin-2-ylmethyl | 410.51 |
| 685 | 4-n-propyloxybenzyl | 417.55 |
| 686 | (S)-(−)-3,7-dimethyloct-6-enyl | 407.59 |
| 687 | 3,4-dichlorobenzyl | 428.36 |
| 688 | 2,4-dimethylbenzyl | 387.52 |
| 689 | 1,4-benzodioxan-6-ylmethyl | 417.50 |
| 690 | 3,5-difluorobenzyl | 395.45 |
| 691 | 3,4-difluorobenzyl | 395.45 |
| 692 | 2-chloro-4-hydroxybenzyl | 409.91 |
| 693 | 2-fluoro-3-trifluoromethylbenzyl | 445.45 |
| 694 | 3-trifluoromethoxybenzyl | 443.46 |
| 695 | 3-bromo-4-methoxybenzyl | 468.39 |
| 696 | 3-methylbenzyl | 373.49 |
| 697 | 3-bromobenzyl | 438.36 |
| 698 | 3,5-dimethoxybenzyl | 419.52 |
| 699 | 2-chloro-4-fluorobenzyl | 411.90 |
| 700 | 5-chloro-thiophen-2-ylmethyl | 499.94 |
| 701 | 2-bromobenzyl | 438.36 |
| 702 | benzofuran-2-ylmethyl | 399.49 |
| 703 | 5-bromo-furan-2-ylmethyl | 428.32 |
| 704 | 4-diethylaminobenzyl | 430.59 |
| 705 | 6-chloro-3,4-methylenedioxybenzyl | 437.92 |
| 706 | 3-bromo-4-fluorobenzyl | 456.35 |
| 707 | 3-chloro-4-fluorobenzyl | 411.90 |
| 708 | 3,5-dichlorobenzyl | 428.36 |
| 709 | 4-n-hexyloxybenzyl | 459.63 |
| 710 | trans-4-stilbene-2-yl-methyl | 461.60 |
| 711 | (R)-(+)-3,7-dimethyloct-6-enyl | 407.59 |
| 712 | 2-carboethoxy-cycloprop-1-ylmethyl | 395.50 |
| 713 | 2,3-dihydrobenzo[b]furan-5-ylmethyl | 401.50 |
| 714 | 5-bromothiophen-2-ylmethyl | 444.39 |
| 715 | 3-(3,4-dichlorophenoxy)benzyl | 520.45 |
| 716 | 3-nitrobenzyl | 404.46 |
| 717 | 3,4,5-trihydroxy-n-pentyl | 387.47 |
| 718 | 3-cyanobenzyl | 384.48 |
| 719 | 4(5)-imidazolylmethyl | 349.43 |
| 720 | 3-hydroxybenzyl | 375.47 |

### EXAMPLE 721

### 1-(2-aminoethyl)-7-(pyridin-4-ylmethyl)-3H,7H-pyrrolo[3,2-f]isoquinolin-6-one

A solution of 2-N-(pyridin-4-ylmethyl)-6-hydrazino-1-isoquinolinone (prepared from 4-bromomethylpyridine according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=354.83; HPLC single component; 1H-NMR, 5.5 δ, 2H, s).

elemental Analysis for: C₁₉H₁₈N₄O 1.0 HCl Calculated; C, 64.31; H, 5.40; N, 15.79 Found: C, 64.47; H, 5.47; N, 15.86

### EXAMPLES 722-806

Using essentially the same procedure described in Example 7B and employing 1-(2-aminoethyl)-7-(pyridin-4-ylmethyl)-3H,7H-pyrrolo[3,2-f]isoquinolin-6-one as substrate and a suitable aldehyde, the compounds shown in Table X were obtained and identified by HPLC and mass spectral analyses.

TABLE X

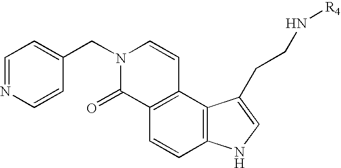

| Ex. | | |
|---|---|---|
| No. | R4 | Mass |
| 722 | naphth-2-ylmethyl | 458.56 |
| 723 | 4-phenoxybenzyl | 500.60 |
| 724 | ethyl | 346.43 |
| 725 | 2-chlorobenzyl | 442.94 |
| 726 | furfur-2-ylmethyl | 398.46 |
| 727 | 4-dimethylaminobenzyl | 451.57 |
| 728 | benzyl | 408.50 |
| 729 | thiophen-2-ylmethyl | 414.53 |
| 730 | 4-methylbenzyl | 422.52 |
| 731 | 4-chlorobenzyl | 442.94 |
| 732 | 4-cyanobenzyl | 433.51 |
| 733 | n-pentyl | 388.51 |
| 734 | 3,4-methylenedioxybenzyl | 452.51 |
| 735 | 4-isopropylbenzyl | 450.58 |
| 736 | 5-methylfurfur-2-ylmethyl | 512.49 |
| 737 | 4-hydroxybenzyl | 424.50 |
| 738 | 4-methoxybenzyl | 438.52 |
| 739 | 2-methoxybenzyl | 438.52 |
| 740 | 3-fluoro-4-methoxybenzyl | 456.51 |
| 741 | 3,5-bis(trifluoromethyl)benzyl | 544.49 |
| 742 | 2,6-difluorobenzyl | 444.48 |
| 743 | 2-fluorobenzyl | 426.49 |
| 744 | 2-trifluoromethylbenzyl | 476.50 |
| 745 | 3-trifluoromethylbenzyl | 476.50 |
| 746 | 4-trifluoromethylbenzyl | 476.50 |
| 747 | 3-fluorobenzyl | 426.49 |
| 748 | 4-fluorobenzyl | 426.49 |
| 749 | indol-3-ylmethyl | 447.53 |
| 750 | furfur-3-ylmethyl | 398.46 |
| 751 | thiophen-3-ylmethyl | 414.53 |
| 752 | pyridin-3-ylmethyl | 409.49 |
| 753 | 2-methylbenzyl | 422.52 |
| 754 | 3-chlorobenzyl | 442.94 |
| 755 | 3-methylbutyl | 388.51 |
| 756 | 3-methoxybenzyl | 438.52 |
| 757 | 4-trifluoromethoxybenzyl | 492.50 |
| 758 | pyridin-4-ylmethyl | 409.49 |
| 759 | 2,4-dichlorobenzyl | 477.39 |
| 760 | pyridin-2-ylmethyl | 409.49 |
| 761 | 6-methyl-pyridin-2-ylmethyl | 423.51 |
| 762 | 4-bromobenzyl | 487.39 |
| 763 | 1-methylpyrrol-2-ylmethyl | 411.50 |
| 764 | 2,4-difluorobenzyl | 444.48 |
| 765 | 4-carbomethoxybenzyl | 466.53 |
| 766 | cyclohexylmethyl | 414.55 |
| 767 | 4-phenylbenzyl | 484.60 |
| 768 | 4-thiomethylbenzyl | 454.59 |
| 769 | 4-ethylbenzyl | 436.55 |
| 770 | quinolin-2-ylmethyl | 459.55 |
| 771 | 4-n-propyloxybenzyl | 466.58 |
| 772 | (S)-(−)-3,7-dimethyloct-6-enyl | 456.63 |
| 773 | 3,4-dichlorobenzyl | 477.39 |
| 774 | 2,4-dimethylbenzyl | 436.55 |
| 775 | 1,4-benzodioxan-6-ylmethyl | 466.53 |
| 776 | 3,5-difluorobenzyl | 444.48 |
| 777 | 3,4-difluorobenzyl | 444.48 |
| 778 | 2-chloro-4-hydroxybenzyl | 458.94 |
| 779 | 2-fluoro-3-trifluoromethylbenzyl | 494.49 |
| 780 | 3-trifluoromethoxybenzyl | 492.50 |
| 781 | 3-bromo-4-methoxybenzyl | 517.42 |
| 782 | 3-methylbenzyl | 422.52 |
| 783 | 3-bromobenzyl | 487.39 |
| 784 | 3,5-dimethoxybenzyl | 468.55 |
| 785 | 2-chloro-4-fluorobenzyl | 460.93 |
| 786 | 5-chloro-thiophen-2-ylmethyl | 448.97 |
| 787 | 2-bromobenzyl | 487.39 |
| 788 | benzofuran-2-ylmethyl | 448.52 |
| 789 | 5-bromo-furan-2-ylmethyl | 477.36 |
| 790 | 4-diethylaminobenzyl | 479.62 |
| 791 | 6-chloro-3,4-methylenedioxybenzyl | 486.95 |
| 792 | 3-bromo-4-fluorobenzyl | 505.38 |
| 793 | 3-chloro-4-fluorobenzyl | 460.93 |
| 794 | 3,5-dichlorobenzyl | 477.39 |
| 795 | 4-n-hexyloxybenzyl | 508.66 |
| 796 | trans-4-stilbene-2-yl-methyl | 510.63 |
| 797 | (R)-(+)-3,7-dimethyloct-6-enyl | 456.63 |
| 798 | 2-carboethoxy-cycloprop-1-ylmethyl | 444.53 |
| 799 | 2,3-dihydrobenzo[b]furan-5-ylmethyl | 450.54 |
| 800 | 5-bromothiophen-2-ylmethyl | 493.42 |
| 801 | 3-(3,4-dichlorophenoxy)benzyl | 569.48 |
| 802 | 3-nitrobenzyl | 453.50 |
| 803 | 3,4,5-trihydroxy-n-pentyl | 436.51 |
| 804 | 3-cyanobenzyl | 433.51 |
| 805 | 4(5)-imidazolylmethyl | 398.46 |
| 806 | 3-hydroxybenzyl | 424.50 |

### EXAMPLE 807

### 1-(2-aminoethyl)-7-(pyridin-2-ylmethyl)-3H,7H-pyrrolo[3,2-f]isoquinolin-6-one

A solution of 2-N-(pyridin-2-ylmethyl)-6-hydrazino-1-isoquinolinone (prepared from 2-bromomethylpyridine according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=354.83; HPLC single component; 1H-NMR, 5.5 δ, 2H, s).

Elemental Analysis for: C₁₉H₈₁N₄O 1.0 HCl Calculated: C, 64.31; H, 5.40; N, 15.79 Found: C, 64.41; H, 5.44; N, 15.80

### EXAMPLES 808-892

Using essentially the same procedure described in Example 7B and employing 1-(2-aminoethyl)-7-(pyridin-2-ylmethyl)-3H,7H-pyrrolo[3,2-f]isoquinolin-6-one as substrate and a suitable aldehyde, the compounds shown in Table XI were obtained and identified by HPLC and mass spectral analyses.

TABLE XI

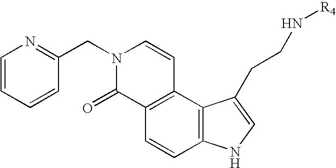

| Ex. | | |
|---|---|---|
| No. | R4 | Mass |
| 808 | naphth-2-ylmethyl | 458.56 |
| 809 | 4-phenoxybenzyl | 500.60 |
| 810 | ethyl | 346.43 |
| 811 | 2-chlorobenzyl | 442.94 |
| 812 | furfur-2-ylmethyl | 398.46 |
| 813 | 4-dimethylaminobenzyl | 451.57 |
| 814 | benzyl | 408.50 |
| 815 | thiophen-2-ylmethyl | 414.53 |
| 816 | 4-methylbenzyl | 422.52 |
| 817 | 4-chlorobenzyl | 442.94 |
| 818 | 4-cyanobenzyl | 433.51 |
| 819 | n-pentyl | 388.51 |
| 820 | 3,4-methylenedioxybenzyl | 452.51 |
| 821 | 4-isopropylbenzyl | 450.58 |
| 822 | 5-methylfurfur-2-ylmethyl | 512.49 |
| 823 | 4-hydroxybenzyl | 424.50 |
| 824 | 4-methoxybenzyl | 438.52 |
| 825 | 2-methoxybenzyl | 438.52 |
| 826 | 3-fluoro-4-methoxybenzyl | 456.51 |
| 827 | 3,5-bis(trifluoromethyl)benzyl | 544.49 |
| 828 | 2,6-difluorobenzyl | 444.48 |
| 829 | 2-fluorobenzyl | 426.49 |
| 830 | 2-trifluoromethylbenzyl | 476.50 |
| 831 | 3-trifluoromethylbenzyl | 476.50 |
| 832 | 4-trifluoromethylbenzyl | 476.50 |
| 833 | 3-fluorobenzyl | 426.49 |
| 834 | 4-fluorobenzyl | 426.49 |
| 835 | indol-3-ylmethyl | 447.53 |
| 836 | furfur-3-ylmethyl | 398.46 |
| 837 | thiophen-3-ylmethyl | 414.53 |
| 838 | pyridin-3-ylmethyl | 409.49 |
| 839 | 2-methylbenzyl | 422.52 |
| 840 | 3-chlorobenzyl | 442.94 |
| 841 | 3-methylbutyl | 388.51 |
| 842 | 3-methoxybenzyl | 438.52 |
| 843 | 4-trifluoromethoxybenzyl | 492.50 |
| 844 | pyridin-4-ylmethyl | 409.49 |
| 845 | 2,4-dichlorobenzyl | 477.39 |
| 846 | pyridin-2-ylmethyl | 409.49 |
| 847 | 6-methyl-pyridin-2-ylmethyl | 423.51 |
| 848 | 4-bromobenzyl | 487.39 |
| 849 | 1-methylpyrrol-2-ylmethyl | 411.50 |
| 850 | 2,4-difluorobenzyl | 444.48 |
| 851 | 4-carbomethoxybenzyl | 466.53 |
| 852 | cyclohexylmethyl | 414.55 |
| 853 | 4-phenylbenzyl | 484.60 |
| 854 | 4-thiomethylbenzyl | 454.59 |
| 855 | 4-ethylbenzyl | 436.55 |
| 856 | quinolin-2-ylmethyl | 459.55 |
| 857 | 4-n-propyloxybenzyl | 466.58 |
| 858 | (S)-(−)-3,7-dimethyloct-6-enyl | 456.63 |
| 859 | 3,4-dichlorobenzyl | 477.39 |
| 860 | 2,4-dimethylbenzyl | 436.55 |
| 861 | 1,4-benzodioxan-6-ylmethyl | 466.53 |
| 862 | 3,5-difluorobenzyl | 444.48 |
| 863 | 3,4-difluorobenzyl | 444.48 |
| 864 | 2-chloro-4-hydroxybenzyl | 458.94 |
| 865 | 2-fluoro-3-trifluoromethylbenzyl | 494.49 |
| 866 | 3-trifluoromethoxybenzyl | 492.50 |
| 867 | 3-bromo-4-methoxybenzyl | 517.42 |
| 868 | 3-methylbenzyl | 422.52 |
| 869 | 3-bromobenzyl | 487.39 |
| 870 | 3,5-dimethoxybenzyl | 468.55 |
| 871 | 2-chloro-4-fluorobenzyl | 460.93 |
| 872 | 5-chloro-thiophen-2-ylmethyl | 448.97 |
| 873 | 2-bromobenzyl | 487.39 |
| 874 | benzofuran-2-ylmethyl | 448.52 |
| 875 | 5-bromo-furan-2-ylmethyl | 477.36 |
| 876 | 4-diethylaminobenzyl | 479.62 |
| 877 | 6-chloro-3,4-methylenedioxybenzyl | 486.95 |
| 878 | 3-bromo-4-fluorobenzyl | 505.38 |
| 879 | 3-chloro-4-fluorobenzyl | 460.93 |
| 880 | 3,5-dichlorobenzyl | 477.39 |
| 881 | 4-n-hexyloxybenzyl | 508.66 |
| 882 | trans-4-stilbene-2-yl-methyl | 510.63 |
| 883 | (R)-(+)-3,7-dimethyloct-6-enyl | 456.63 |
| 884 | 2-carboethoxy-cycloprop-1-ylmethyl | 444.53 |
| 885 | 2,3-dihydrobenzo[b]furan-5-ylmethyl | 450.54 |
| 886 | 5-bromothiophen-2-ylmethyl | 493.42 |
| 887 | 3-(3,4-dichlorophenoxy)benzyl | 569.48 |
| 888 | 3-nitrobenzyl | 453.50 |
| 889 | 3,4,5-trihydroxy-n-pentyl | 436.51 |
| 890 | 3-cyanobenzyl | 433.51 |
| 891 | 4(5)-imidazolylmethyl | 398.46 |
| 892 | 3-hydroxybenzyl | 424.50 |

### EXAMPLE 893

### 1-(2-aminoethyl)-7-(2-dimethylaminoethyl)-3H,7H-Pyrrolo[3,2-]isoquinolin-6-one

A solution of 2-N-(2-dimethylaminoethyl)-6-hydrazino-1-isoquinolinone (prepared from 2-bromo-N,N-dimethylamine according to Example 92 and Example 93) was reacted in a Fisher indole reaction with 2-(3-chloropropyl)-1,3-dioxolan according to the process outlined in Example 4. After refluxing for four hours the product was isolated as reported above and purified by flash silica gel chromatography (20% MeOH, 5% NH₄OH in CH₂Cl₂) to afford a light brown oil. Treatment with ethanolic HCl gave the required product as an off white colored solid (MW=370.30; HPLC single component; IH-NMR, 4.4 δ, 2H, t; 3.35 δ, 6H, s).

Elemental Analysis for: C₁₇H₂₂N₄O 2.0 HCl Calculated: C, 54.99; H, 6.52; N, 15.09 Found: C, 55.17; H, 6.67; N, 15.16

### EXAMPLES 894-978

Using essentially the same procedure described in Example 7B and employing 1-(2-aminoethyl)-7-(2-dimethylaminoethyl)-3H,7H-pyrrolo[3,2-f]isoquinolin-6-one as substrate and a suitable aldehyde, the compounds shown in Table XII were obtained and identified by HPLC and mass spectral analyses.

### EXAMPLE 979

### 1-(2-aminoethyl)-7-(2-methoxybenzyl)-3H,7H-pyrrolo[3,2-f]isoquinolin-6-one

A solution of 2-N-(2-methoxybenzyl)-6-hydrazino-1-isoquinolinone (prepared from 2-methoxybenzylchloride according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=383.87; HPLC single component; 1H-NMR, 3.8 δ, 3H, s; 5.2 δ, 2H, s).

elemental Analysis for: C₂₁H₂₁N₃O₂ 1.0 HCl Calculated; C, 65.71; H, 5.78; N, 10.95 Found; C, 65.71; H, 5.87; N, 11.16

### EXAMPLES 980-987

Using essentially the same procedure described in Example 7B and employing 1-(2-aminoethyl)-7-(2-methoxybenzyl)-3H,7H-pyrrolo[3,2-f]isoquinolin-6-one as substrate and a suitable aldehyde, the compounds shown in Table XIII were obtained and identified by HPLC and mass spectral analyses.

TABLE XIII

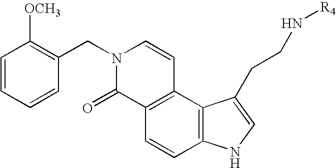

| Ex. | | |
|---|---|---|
| No. | R4 | Mass |
| 980 | 3-nitrobenzyl | 482.53 |
| 981 | 3,4,5-trihydroxy-n-pentyl | 465.54 |
| 982 | 3-chlorobenzyl | 471.98 |
| 983 | 3-methoxybenzyl | 467.56 |
| 984 | pyridin-4-ylmethyl | 438.52 |
| 985 | 3-cyanobenzyl | 462.55 |
| 986 | 4(5)-imidazolylmethyl | 427.50 |
| 987 | 3-hydroxybenzyl | 453.54 |

### EXAMPLE 988

### 1-(2-aminoethyl)-7-(4-methoxybenzyl)-3H,7H-pyrrolo[3,2-f]isoquinolin-6-one

A solution of 2-N-(4-methoxybenzyl)-6-hydrazino-1-isoquinolinone (prepared from 4-methoxybenzylchloride according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=383.87; HPLC single component; 1H-NMR, 3.7 δ, 3H, s; 5.2 δ, 2H, s).

Elemental Analysis for: C₂₁H₂₁N₃O 2 1.0 HCl Calculated: C, 65.71; H, 5.78; N, 10.95 Found: C, 65.79; H, 5.86; N, 11.05

### EXAMPLES 989-996

Using essentially the same procedure described in Example 7B and employing 1-(2-aminoethyl)-7-(4-methoxybenzyl)-3H,7H-pyrrolo[3,2-f]isoquinolin-6-one as substrate and a suitable aldehyde, the compounds shown in Table XIV were obtained and identified by HPLC and mass spectral analyses.

TABLE XIV

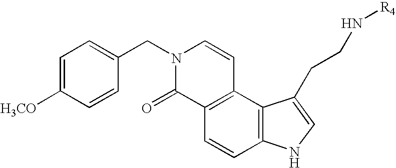

| Ex. | | |
|---|---|---|
| No. | R4 | Mass |
| 989 | 3-nitrobenzyl | 482.53 |
| 990 | 3,4,5-trihydroxy-n-pentyl | 465.54 |
| 991 | 3-chlorobenzyl | 471.98 |
| 992 | 3-methoxybenzyl | 467.56 |
| 993 | pyridin-4-ylmethyl | 438.52 |
| 994 | 3-cyanobenzyl | 462.55 |
| 995 | 4(5)-imidazolylmethyl | 427.50 |
| 996 | 3-hydroxybenzyl | 453.54 |

### EXAMPLE 997

### 1-(2-aminoethyl)-7-(furanyl-2-ylmethyl)-3H,7H-pyrrolo[3,2-f]isoquinolin-6-one [furanyl-2-yl or furan-2-yl?]

A solution of 2-N-(furanyl-2-ylmethyl)-6-hydrazino-1-isoquinolinone (prepared from furfuryl bromide according to Example 92 and Example 93) was reacted in a Fisher indole reaction with 2-(3-chloropropyl)-1,3-dioxolan according to the process outlined in Example 4. After refluxing for four hours, the product was isolated as reported above and purified by flash silica gel chromatography (20% MeOH, 5% NH₄OH in CH₂Cl₂) to afford a light brown oil. Treatment with ethanolic HCl gave the required product as its mono hydrochloride salt as an off white colored solid (MW=343.81; HPLC single component; 1H-NMR, 6.3 δ, 2H, s; 5.2 δ, 2H. s).

Elemental Analysis for: C₁₈H₁₈C1N₃O₂ 1.0 HCl Calculated: C, 62.88; H, 5.28; N, 12.22 Found: C, 62.89; H, 5.36; N, 12.35

### EXAMPLES 998-1005

Using essentially the same procedure described in Example 7B and employing 1-(2-aminoethyl)-7-(furan-2-ylmethyl)-3H,7H-pyrrolo[3,2-f]isoquinolin-6-one as substrate and a suitable aldehyde, the compounds shown in Table XV were obtained and identified by HPLC and mass spectral analyses.

TABLE XV

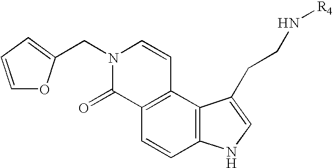

| Ex. | | |
|---|---|---|
| No. | R4 | Mass |
| 998 | 3-nitrobenzyl | 442.47 |
| 999 | 3,4,5-trihydroxy-n-pentyl | 425.48 |
| 1000 | 3-chlorobenzyl | 431.92 |
| 1001 | 3-methoxybenzyl | 427.50 |
| 1002 | pyridin-4-ylmethyl | 398.46 |
| 1003 | 3-cyanobenzyl | 422.48 |
| 1004 | 4(5)-imidazolylmethyl | 387.44 |
| 1005 | 3-hydroxybenzyl | 413.47 |

### EXAMPLES 1006-1017

### 1-(2-benzylaminoethyl)-substituted-pyrrolo[3,2-f]isoquinolin-6-one Compounds

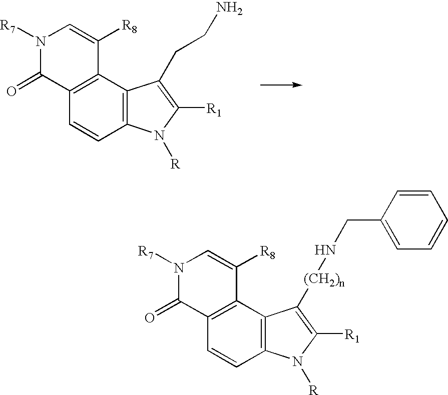

Using the essentially the same procedure described in Example 7B and employing the appropriate 1-(2-aminoethyl)-pyrrolo[3,2-f]isoquinolin-6-one substrate and benzaldehyde, the compounds shown in Table XVI were prepared and identified by HPLC and mass spectral analyses.

TABLE XVI

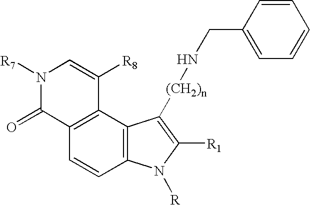

| Ex. | | | | | | |
|---|---|---|---|---|---|---|
| No. | n | R | R1 | R7 | R8 | Mass |
| 1006 | 2 | H | Br | H | H | 432.74 |
| 1007 | 2 | CH₃ | H | benzyl | H | 457.99 |
| 1008 | 2 | H | H | phenethyl | H | 457.99 |
| 1009 | 2 | H | H | naphth-2-ylmethyl | H | 508.05 |
| 1010 | 2 | H | H | heptyl | H | 452.03 |
| 1011 | 2 | H | H | 3,4-dichlorobenzyl | H | 512.86 |
| 1012 | 2 | H | H | thiophen-3-yl | H | 450.00 |
| 1013 | 3 | H | H | H | H | 367.87 |
| 1014 | 2 | H | 4-Cl—C₆H₅ | H | H | 478.41 |
| 1015 | 2 | H | H | H | Cl | 388.29 |
| 1016 | 2 | H | H | tetrahydropyran-2- | H | 451.99 |
| | | | | ylmethyl | | |
| 1017 | 2 | H | H | 2-methoxyethyl | H | 411.92 |

### EXAMPLE 1018

### 3,7-dibenzyl-1-(2-dibenzylaminoethyl)-3H,7H-pyrrolo[3,2-f]isoquinolin-6-one

Tetrabutylammonium bromide (0.03 g) and benzyl bromide (0.3 g, 1.6 mmol) were added to a solution of the amine from Example 4 (0.1 g, 0.44 mmol) in toluene (2 mL) and 50% aqueous sodium hydroxide (1.8 mmol, 4 equivalents). The mixture was stirred for three hours at room temperature, and the product was purified by flash silica gel chromatography (1% methanolic ammonia in CH₂Cl₂) to afford a brown colored solid. Treatment with ethanolic HCl afforded the salt of the titled compound as an off white colored solid (mp 234-236° C.; MW=624.21; HPLC single component).

Elemental Analysis for: C₄₁H₃₇N₃O 1.0 HCl Calculated: C, 78.89; H, 6.14; N, 6.73 Found: C, 78.91; H, 6.23; N, 6.84

### EXAMPLE 1019

### 1-(2-dibenzylaminoethyl)-3H,7H-pyrrolo[3,2-f]isoquinolin-6-one

Benzyl bromide (0.226 g, 1.32 mmol) was added to a solution of the amine from Example 4 (0.2 g, 0.88 mmol) in DMF (2 mL) and 50% aqueous sodium hydroxide (1.75 mmol, 2 equivalents). The mixture was stirred for two hours at room temperature, and the product was purified by flash silica gel chromatography (4% methanolic ammonia in CH₂Cl₂). Treatment with ethanolic HCl afforded the salt of the titled compound as a white solid (mp 250-253° C.; MW=624.21; HPLC single component).

Elemental Analysis for: C₂₇H₂₅N₃O 1.0 HCl Calculated: C, 73.04; H, 5.90; N, 9.46 Found: C, 73.20; H, 5.99; N, 9.56

### EXAMPLE 1020

### 1-[2-(benzyl-methylamino)ethyl]-3H,7H-pyrrolo[3,2-f]isoquinolin-6-one

A solution of 6-hydrazino-1-isoquinolinone (0.4 g, 1.89 mmol) from Example 3 and 2-(3-(N-methyl-benzylamine)propyl)-1,3-dioxolan (0.668 g, 2.84 mmol) in degassed ethanol (120 ml)/4% aqueous sulfuric acid (12 ml) was refluxed under nitrogen for six hours. The reaction mixture was concentrated in vacuo and the product purified by flash silica gel chromatography to afford an orange colored solid (0.155 g, 25% yield). Treatment of the product with ethereal [v. ethanolic?] HCl and crystallization from ethanol ether gave the mono hydrochloride salt as a light amber colored solid (mp=152-155° C.; MW=367.87; HPLC single component).

Elemental Analysis for: C₂₁H₂₁N₃O 1.0 HCl Calculated: C, 68.56; H, 6.03; N, 11.42 Found: C, 88.62; H, 6.12; N, 11.54

### EXAMPLE 1021

### 1-(2-methylaminoethyl)-3H,7H-pyrrolo[3,2-f]isoquinolin-6-one

1-[2-(Benzylmethylamino)ethyl]-3H,7H-pyrrolo[3,2-f]isoquinolin-6-one (1.07 g, 3.22 mmol) from Example 1020 was hydrogenated over 10% Pd/C at 50 psi H₂ for 24 hours. The catalyst was removed by filtration, and the product was converted to its HCl salt by the action of ethanolic HCl. Recrystallization from EtOH/Et₂O afforded the titled compound as a light brown colored solid (mp=160° C.; MW=377.75; HPLC single component).

Elemental Analysis for: C₁₄H₁₅N₃O 1.0 HCl Calculated: C, 60.54; H, 5.81; N, 15.13 Found: C, 60.64; H, 5.98; N, 15.24

### EXAMPLE 1022

### N-[2-(7-benzyl-6-oxo-6,7-dihydro-3H-pyrrolo[3,2-f]isoquinolin-1-yl)-ethyl]-benzenesulfonamide

A solution of 1-(2-aminoethyl)-7-benzyl-3H,7H-pyrrolo[3,2-f]isoquinolin-6-one (0.25 g, 0.788 mmol) from Example 182 in DMF (4 mL) was treated with sodium hydride (1.2 equivalents) at 0° C. followed after 15 minutes with the addition of benzene sulfonylchloride (0.18 g, 1.02 mmol, 1.3 equivalents). The mixture was stirred for 16 hours and the titled compound isolated as a white solid (m.p. 205-206° C.; MW=457.55; HPLC single component) by flash silica gel chromatography (5% MeOH in CH₂Cl₂).

Elemental Analysis for: C₂₆H₂₃N₃O₃S Calculated: C, 68.25; H, 5.07; N, 9.18 Found: C, 68.33; H, 5.18; N, 9.27

### EXAMPLE 1023

### 7-fluoro-3H-quinazolin-4-one

A solution of 2-amino-4-fluorobenzoic acid (8.8 g, 56.7 mmol) and formamidine acetate (23.63 g, 227 mmol, 4 equivalents) in ethoxyethanol (400 ml) was refluxed for 16 hours. The solvent was removed in vacuo and the residue crystallized from methanol-water to provide the titled compound as a light brown solid (8.35 g, 90% yield, M+1=165).

Elemental Analysis for: C₈H₅FN₂O Calculated: C, 58.54; H, 3.07; N, 17.07 Found: C, 58.60; H, 3.19; N, 17.27

### EXAMPLE 1024

### 7-fluoro-3-methyl-3H-quinazolin-4-one

Sodium hydride (19.0 mmol, 1.2 eq) was added to a solution of 7-fluoro-3H-quinazolin-4-one (2.6 g, 15.8 mmol, Example 1023) in DMF (30 ml) under an atmosphere of nitrogen at −20° C. After stirring for one hour, methyl iodide (19.8 mmol, 1.25 equivalents) was added, and the resulting solution was stirred at room temperature for two hours. Water (100 ml) was added and the product extracted into CH₂Cl₂ (3×100 ml). The organics were concentrated in vacuo and the product crystallized from hexane/CH₂Cl₂ to afford the titled compound as a light amber colored solid (m.p. 133-135° C., M+1=179).

Elemental Analysis for: C₉H₇FN₂O Calculated: C, 60.67; H, 3.96; N, 15.72 Found: C, 60.72; H, 3.99; N, 15.79

### EXAMPLE 1025

### 7-hydrazino-3-methyl-3H-quinazolin-4-one

A solution containing 7-fluoro-3-methyl-3H-quinazolin-4-one (2.2 g, 12.3 mmol, Example 1026) and hydrazine (7.88 g, 20 equivalents) in dioxane (50 ml) was refluxed for 16 hours under an atmosphere of nitrogen. The mixture was concentrated in vacuo, triturated with dioxane and the resulting solid washed with water (50 ml) and dried under vacuum to provide the titled compound as a white solid (1.63 g, 70% yield, m.p. 208-211° C., M+1=191).

Elemental Analysis for: C₉H₁₀N₄O Calculated: C, 56.83; H, 5.30; N, 29.46 Found: C, 56.98; H, 5.39; N, 29.59

### EXAMPLE 1026

### 3-benzyl-7-fluoro-3H-quinazolin-4-one

Sodium hydride (22.0 mmol, 1.2 equivalents) was added to a solution of 7-fluoro-3H-quinazolin-4-one (3.0 g, 18.3 mmol, Example 1023) in DMF (40 ml) under an atmosphere of nitrogen at −20° C. After stirring for one hour, benzyl bromide (3.7 g, 22 mmol, 1.2 equivalents) was added, and the resulting solution was stirred at room temperature for two hours. Water (100 ml) was added and the product extracted into CH₂Cl₂ (3×100 ml). The organics were concentrated in vacuo and the product crystallized from hexane/CH₂Cl₂ to afford the titled compound as a light brown/beige colored solid (m.p. 110-111° C., M+1=255).

Elemental Analysis for: C₉H₇FN₂O Calculated: C, 70.86; H, 4.36; N, 11.02 Found: C, 70.94; H, 4.39; N, 11.19

### EXAMPLE 1027

### 3-benzyl-7-hydrazino-3H-quinazolin4-one

A solution containing 3-benzyl-7-fluoro-3H-quinazolin-4-one (3.0 g, 11.8 mmol) and hydrazine (7.56 g, 20 equivalents) in dioxane (60 ml) was refluxed for 16 hours under an atmosphere of nitrogen. The mixture was concentrated in vacuo, and the resulting solid crystallized from methanol/water to provide the titled compound as a cream colored solid (2.4 g, 76% yield, m.p. 165-169° C., M+1=267).

Elemental Analysis for: C₁₅H₁₄N₄O Calculated: C, 67.65; H, 5.30; N, 21.04 Found: C, 67.78; H, 5.41; N, 21.19

### EXAMPLE 1028

### 9-(2-aminoethyl)-3-methyl-3H,7H-pyrrolo[2,3-h]quinazolin-4-one

A solution of 7-hydrazino-3-methyl-3H-quinazolin-4-one (1.48 g, 7.78 mmol), sulfuric acid (1N, 16 ml) and 2-(3-chloropropyl)-1,3-dioxolan (1.41 g, 9.34 mmol) in degassed ethanol (50 ml)/water (50 ml) was refluxed under nitrogen for three hours. The reaction mixture was concentrated in vacuo and the product purified by flash silica gel chromatography, eluting with Et₃N (2%): MeOH (20%): CH₂Cl₂ (78%), affording an amber colored solid (1.245 g). The product was crystallized from ethanol to afford the titled compound as a light amber colored solid (m.p. 279-285° C., M+1=243).

Elemental Analysis for: C₁₃H₁₄N₄O Calculated: C, 64.45; H, 5.82; N, 23.12 Found: C, 64.58; H, 5.89; N, 23.19

### EXAMPLES 1029-1032

### 9-(2-Aminoethyl)-substituted-pyrrolo[2,3-h]quinazolin-4-one Compounds

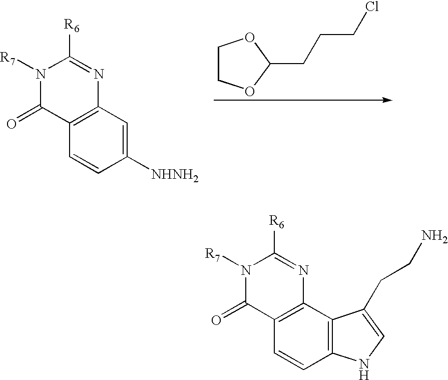

Using the essentially the same procedure described in Example 1028 and employing the appropriate 7-hydrazino-3H-quinazolin-4-one substrate and 2-(3-chloropropyl)-1,3-dioxolan, the compounds shown in Table XVII were prepared and identified by HPLC and mass spectral analyses.

TABLE XVII

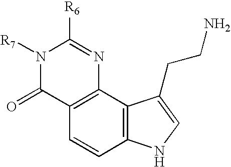

| Ex. | | | mp | |
|---|---|---|---|---|
| No. | R6 | R7 | ° C. | [M + H] |
| 1029 | H | benzyl | 302-305 | 319 |
| 1030 | CH₃ | CH₃ | — | — |
| 1031 | 3-pyridinyl | H | — | — |
| 1032 | H | H | — | — |

### EXAMPLE 1033

Comparative Evaluation of 5-HT₆ Binding Affinity of Test Compounds

The affinity of test compounds for the 5-HT₆ receptor is evaluated in the following manner. Cultured Hela cells expressing human cloned 5-HT₆ receptors are harvested and centrifuged at low speed (1,000×g) for 10.0 min to remove the culture media. The harvested cells are suspended in half volume of fresh physiological phosphate buffered saline (PBS) solution and recentrifuged at the same speed. This operation is repeated. The collected cells are then homogenized in ten volumes of 50 mM Tris.HCl (pH 7.4) and 0.5 mM EDTA. The homogenate is centrifuged at 40,000×g for 30.0 min, and the precipitate is collected. The obtained pellet is resuspended in 10 volumes of Tris.HCl buffer and recentrifuged at the same speed. The final pellet is suspended in a small volume of Tris.HCl buffer, and the tissue protein content is determined in aliquots of 10-25 microliter volumes. Bovine Serum Albumin is used as the standard in the protein determination according to the method described in Lowry et al., J. Biol. Chem. 1951, 193, 265. The volume of the suspended cell membranes is adjusted to give a tissue protein concentration of 1.0 mg/ml of suspension. The prepared membrane suspension (10 times concentrated) is aliquoted in 1.0 ml volumes and stored at −70° C. until used in subsequent binding experiments.

Binding experiments are performed in a 96-well microtiter plate format, in a total volume of 200 microliters. To each well is added the following mixture: 80.0 microliter of incubation buffer made in 50 mM Tris.HCl buffer (pH 7.4) containing 10.0 mM MgCl₂ and 0.5 mM EDTA and 20 microliters of [³H]-LSD (S.A., 86.0 Ci/mmol, available from Amersham Life Science), 3.0 nM. The dissociation constant, K_{D} of the [³H]-LSD at the human 5-HT₆ receptor is 2.9 nM, as determined by saturation binding with increasing concentrations of [³H]-LSD. The reaction is initiated by the final addition of 100.0 microliters of tissue suspension. Nonspecific binding is measured in the presence of 10.0 micromoles methiothepin. The test compounds are added in 20.0 microliter volume.

The reaction is allowed to proceed in the dark for 120 min at room temperature, at which time, the bound ligand-receptor complex is filtered off on a 96-well unifilter with a Packard Filtermate® 196 Harvester. The bound complex caught on the filter disk is allowed to air dry, and the radioactivity is measured in a Packard TopCount® equipped with six photomultiplier detectors, after the addition of 40.0 micoliters Micorscint®-20 scintillant to each shallow well. The unifilter plate is heat-sealed and counted in a Packard TopCount® with a tritium efficiency of 31%.

Specific binding to eh 5-HT₆ receptor is defined as the total radioactivity bound less the amount bound in the presence of 10.0 microliter unlabelled methiothipin. Binding in the presence of varying concentrations of test compound is expressed as a percentage of specific binding in the absence of test compound. The results are plotted as log % bound versus log concentration of the test compound. Nonlinear regression analysis of data points with a computer assisted program Prism® yielded both the IC₅₀ and the Kᵢ values of the test compounds with 95% confidence limits. A linear regression is plotted, from which the IC₅₀ value is determined, and the Kᵢ value is determined based upon the following equation:
Kᵢ=IC₅₀/(1+L/K_{D})
where L is the concentration of the radioactive ligand used and K_{D} is the dissociation constant of the ligand for the receptor, both expressed in nM.

Using this assay, the Kᵢ values were determined and are shown in Table XVIII below.

TABLE XVIII

| Test Compound | 5-HT6 binding Ki |
|---|---|
| (Ex. No.) | (nM) |
| 7 | 15 |
| 94 | 18 |
| 182 | 11 |
| 269 | 13 |
| 274 | 11 |
| 1021 | 21 |
| 1029 | 32.0 |
| Comparative | 5-HT6 Binding Ki |
| Examples | (nM) |
| loxapine | 41.4 |
| bromocriptine | 23.0 |
| methiothepin | 8.3 |
| mianserin | 44.2 |
| olanzepine | 19.5 |

What is claimed is:

1. A compound of formula I 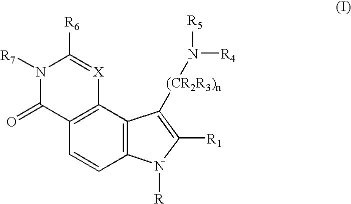
wherein
X is N or CR₈;
n is an integer of 1, 2, 3, 4, 5, or 6;
R is H, SO₂R₉ or an alkyl, cycloalkyl, alkenyl or alkynyl group each optionally substituted;
R₁ is H, halogen or an alkyl, aryl or heteroaryl group each optionally substituted;
R₂ and R₃ are each independently H or an optionally substituted alkyl group;
R₄ and R₅ are each independently H or an alkyl, alkenyl, cycloalkyl, cycloheteroalkyl, aryl or heteroaryl group each optionally substituted, or R₄ and R₅ may be taken together with the atom to which they are attached to form an optionally substituted 5- to 8-membered ring optionally containing an additional heteroatom selected from O, S or NR₇;
R₆ and R₈ are each independently H, halogen or an alkyl, alkenyl, alkynyl, alkoxy, cycloalkyl, cycloheteroalkyl, aryl or heteroaryl group each optionally substituted;
R₇ is H or an alkyl, alkenyl, alkynyl, cycloalkyl, cycloheteroalkyl, aryl, or heteroaryl group each optionally substituted; and
R₉ is an alkyl, aryl or heteroaryl group each optionally substituted; or a stereoisomer thereof or a pharmaceutically acceptable salt thereof.

2. The compound according to claim 1 wherein n is 2.

3. The compound according to claim 1 wherein R is H.

4. The compound according to claim 1 wherein R₇ is H or an optionally substituted alkyl group.

5. The compound according to claim 2 wherein R₁ is H.

6. The compound according to claim 2 wherein R₂ and R₃ are H.

7. The compound according to claim 2 wherein R is H and R₇ is H or an optionally substituted alkyl group.

8. The compound according to claim 7 wherein R₁, R₂ and R₃ are H.

9. The compound according to claim 1 selected from the group consisting essentially of:
1-(2-benzylaminoethyl)-3H,7H -pyrrolo[3,2-f]isoquinolin-6-one;
1-(2-aminoethyl)-3H,7H-pyrrolo[3,2-f]isoquinolin-6-one;
1-(2-aminoethyl)-2-bromo-3H,7H-pyrrolo[3,2-f]isoquinolin-6-one;
1-(2-aminoethyl)-7-methyl-3H,7H-pyrrolo[3,2-f]isoquinolin-6-one;
1-(2-aminoethyl)-7-benzyl-3H,7H-pyrrolo[3,2-f]isoquinolin-6-one;
1-(2-aminoethyl)-7-cyclohexylmethyl-3H,7H-pyrrolo[3,2-f]isoquinolin-6-one;
1-(2-aminoethyl)-7-thien-3-ylmethyl-3H,7H-pyrrolo[3,2-f]isoquinolin-6-one;
1-(2-aminoethyl)-7-(furan-2-ylmethyl)-3H,7H-pyrrolo[3,2-f]isoquinolin-6-one;
1-(2-methylaminoethyl)-3H,7H-pyrrolo[3,2-f]isoquinolin-6-one;
9-(2-aminoethyl)-3-benzyl-3H,7H-pyrrolo[2,3-h]quinazolin-4-one;
9-(2-aminoethyl)-3-methyl-3H,7H-pyrrolo[2,3-h]quinazolin-4-one;
9-(2-aminoethyl)-3-benzyl-3H,7H-pyrrolo[2,3-h]quinazolin-4-one;
9-(2-aminoethyl)-3H,7H-pyrrolo[2,3-h]quinazolin4-one;
9-(2-aminoethyl)-2-(3-pyridinyl)-3H,7H-pyrrolo[2,3-h]quinazolin4-one;
9-(2-aminoethyl)-2,3-dimethyl-3H,7H-pyrrolo[2,3-]isoquinazolin-4-one;
a stereoisomer thereof; and
a pharmaceutically acceptable salt thereof.

10. A method for the treatment of a central nervous system disorder related to or affected by the 5-HT6 receptor in a patient in need thereof which comprises providing to said patient a therapeutically effective amount of a compound of formula I 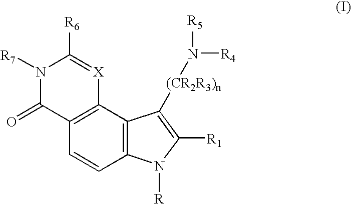
wherein
X is N or CR₈;
n is an integer of 1, 2, 3, 4, 5, or 6;
R is H, SO₂R₉ or an alkyl, cycloalkyl, alkenyl or alkynyl group each optionally substituted;
R₁ is H, halogen or an alkyl, aryl or heteroaryl group each optionally substituted;
R₂ and R₃ are each independently H or an optionally substituted alkyl group;
R₄ and R₅ are each independently H or an alkyl, alkenyl, cycloalkyl, cycloheteroalkyl, aryl or heteroaryl group each optionally substituted, or R₄ and R₅ may be taken together with the atom to which they are attached to form an optionally substituted 5- to 8-membered ring optionally containing an additional heteroatom selected from O, S or NR₇;
R₆ and R₈ are each independently H, halogen or an alkyl, alkenyl, alkynyl, alkoxy, cycloalkyl, cycloheteroalkyl, aryl or heteroaryl group each optionally substituted;
R₇ is H or an alkyl, alkenyl, alkynyl, cycloalkyl, cycloheteroalkyl, aryl, or heteroaryl group each optionally substituted; and
R₉ is an alkyl, aryl or heteroaryl group each optionally substituted; or a stereoisomer thereof or a pharmaceutically acceptable salt thereof.

11. The method according to claim 10 wherein said disorder is an anxiety disorder or a cognitive disorder.

12. The method according to claim 10 wherein said disorder is a neurodegenerative disorder.

13. The method according to claim 10 wherein said disorder is selected from the group consisting essentially of: schizophrenia; attention deficit disorder; obsessive compulsive disorder; withdrawal from drug, alcohol or nicotine addiction; depression; and Alzheimer's disease.

14. The method according to claim 12 wherein said disorder is stroke, head trauma or neuropathic pain.

15. A pharmaceutical composition which comprises a pharmaceutically acceptable carrier and an effective amount of a compound of formula I 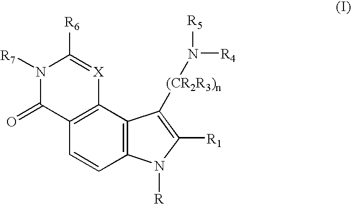
wherein
X is N or CR₈;
n is an integer of 1, 2, 3, 4, 5, or 6;
R is H, SO₂R₉ or an alkyl, cycloalkyl, alkenyl or alkynyl group each optionally substituted;
R₁ is H, halogen or an alkyl, aryl or heteroaryl group each optionally substituted;
R₂ and R₃ are each independently H or an optionally substituted alkyl group;
R₄ and R₅ are each independently H or an alkyl, alkenyl, cycloalkyl, cycloheteroalkyl, aryl or heteroaryl group each optionally substituted, or R₄ and R₅ may be taken together with the atom to which they are attached to form an optionally substituted 5- to 8-membered ring optionally containing an additional heteroatom selected from O, S or NR₇;
R₆ and R₈ are each independently H, halogen or an alkyl, alkenyl, alkynyl, alkoxy, cycloalkyl, cycloheteroalkyl, aryl or heteroaryl group each optionally substituted;
R₇ is H or an alkyl, alkenyl, alkynyl, cycloalkyl, cycloheteroalkyl, aryl, or heteroaryl group each optionally substituted; and
R₉ is an alkyl, aryl or heteroaryl group each optionally substituted; or a stereoisomer thereof or a pharmaceutically acceptable salt thereof.

16. The composition according to claim 15 having a formula I compound wherein n is 2.

17. The composition according to claim 16 having a formula I compound wherein R and R₁ are H.

18. The composition according to claim 17 having a formula I compound wherein R₇ is H or an optionally substituted alkyl group.

19. The composition according to claim 18 having a formula I compound wherein R₂ and R₃ are H and R₄ and R5 each independently H or an optionally substituted alkyl group.

20. The composition according to claim 10 having a formula I compound selected from the group consisting essentially of:
1-(2-benzylaminoethyl)-3H,7H -pyrrolo[3,2-f]isoquinolin-6-one;
1-(2-aminoethyl)-3H,7H-pyrrolo[3,2-f]isoquinolin-6-one;
1-(2-aminoethyl)-2-bromo-3H,7H-pyrrolo[3,2-f]isoquinolin-6-one;
1-(2-aminoethyl)-7-methyl-3H,7H-pyrrolo[3,2-f]isoquinolin-6-one;
1-(2-aminoethyl)-7-benzyl-3H,7H-pyrrolo[3,2-f]isoquinolin-6-one;
1-(2-aminoethyl)-7-cyclohexylmethyl-3H,7H-pyrrolo[3,2-]isoquinolin-6-one;
1-(2-aminoethyl)-7-thien-3-ylmethyl-3H,7H-pyrrolo[3,2-f]isoquinolin-6-one;
1-(2-aminoethyl)-7-(furan-2-ylmethyl)-3H,7H-pyrrolo[3,2-f]isoquinolin-6-one;
1-(2-methylaminoethyl)-3H,7H-pyrrolo[3,2-f]isoquinolin-6-one;
9-(2-aminoethyl)-3-benzyl-3H,7H-pyrrolo[2,3-h]quinazolin-4-one;
9-(2-aminoethyl)-3-methyl-3H,7H-pyrrolo[2,3-h]quinazolin-4-one;
9-(2-aminoethyl)-3-benzyl-3H,7H-pyrrolo[2,3-h]quinazolin4-one;
9-(2-aminoethyl)-3H,7H-pyrrolo[2,3-h]quinazolin-4-one;
9-(2-aminoethyl)-2-(3-pyridinyl)-3H,7H-pyrrolo[2,3-h]quinazolin-4-one;
9-(2-aminoethyl)-2,3-dimethyl-3H,7H-pyrrolo[2,3-h]quinazolin-4-one;
a stereoisomer thereof; and
a pharmaceutically acceptable salt thereof.
